(19) United States
(12) Patent Application Publication
(10) Pub. No.: US 2025/0223587 A1
(43) Pub. Date: Jul. 10, 2025

(54) COMPOSITIONS AND METHODS FOR TREATING A CONGENITAL EYE DISEASE

(71) Applicant: Beam Therapeutics Inc., Cambridge, MA (US)

(72) Inventors: David Bryson, Cambridge, MA (US); Jack Sullivan, Cambridge, MA (US)

(73) Assignee: Beam Therapeutics Inc., Cambridge, MA (US)

(21) Appl. No.: 19/034,326

(22) Filed: Jan. 22, 2025

Related U.S. Application Data
(63) Continuation of application No. PCT/US2023/071249, filed on Jul. 28, 2023.
(60) Provisional application No. 63/369,881, filed on Jul. 29, 2022.

(51) Int. Cl. C12N 15/11; A61K 48/00; C12N 9/78; C12N 15/10

(52) U.S. Cl. CPC C12N 15/11; A61K 48/005; C12N 9/78; C12N 15/102; C12Y 305/04005; C12N 2310/20; C12N 2320/33; C12N 2320/34

(57) ABSTRACT

The present disclosure features compositions and methods for editing a 290-KD centrosomal protein (CEP290) gene to treat a congenital eye disorder, such as Leber's Congenital Amaurosis-10. In embodiments, the disclosure provides methods for direct correction of the IVS26 pathogenic mutation in the CEP290 gene (CEP290 c.2991+1655A>G) and/or disruption of a cryptic splice donor site within an intron of the CEP290 gene using a base editor (e.g., a cytidine deaminase base editor, an adenosine deaminase base editor, or a cytidine adenosine deaminase base editor (CABE)).

### CROSS REFERENCE TO RELATED APPLICATIONS

This application is a continuation under 35 U.S.C. § 111(a) of PCT International Patent Application No. PCT/US2023/071249, filed Jul. 28, 2023, designating the United States and published in English, which claims priority to and the benefit of U.S. Provisional Application No. 63/369,881, filed Jul. 29, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Mar. 28, 2025, is named 180802-055203US_SL.xml and is 1,006,640 bytes in size.

### BACKGROUND

Leber's Congenital Amaurosis-10 (LCA10) is a rare congenital eye disease that appears at birth or in the first few months of life. It affects about 1 in 40,000 newborns. The disease primarily affects the retina. People with the disease typically have severe visual impairment beginning in infancy, and the impairment can worsen progressively over time. Typically, LCA10 leads to progressive loss of all vision. There is a need for improved compositions and methods for treating LCA10.

### SUMMARY

As described below, the invention features compositions and methods for editing a 290-KD centrosomal protein (CEP290) gene to treat a congenital eye disorder, such as Leber's Congenital Amaurosis-10 (LCA10). In embodiments, the disclosure provides methods for direct correction of the IVS26 pathogenic mutation in the CEP290 gene (CEP290 c.2991+1655A>G) and/or disruption of a cryptic splice donor site within an intron of the CEP290 gene using a base editor (e.g., a cytidine deaminase base editor, an adenosine deaminase base editor, or a cytidine adenosine deaminase base editor (CABE)).

In one aspect, the invention features a method of editing a nucleobase of a 290-KD centrosomal protein (CEP290) polynucleotide in a cell. The method involves contacting the cell with a base editor polypeptide containing (a) a nucleic acid programmable DNA binding protein (napDNAbp) domain and a deaminase domain, or (b) one or more polynucleotides encoding the base editor. The method further involves contacting the cell with one or more guide polynucleotides, or one or more polynucleotides encoding the one or more guide polynucleotides, where the guide polynucleotides target the base editor to effect an alteration of the nucleobase of the CEP290 polynucleotide in the cell.

In another aspect, the invention features a method of treating Leber's Congenital Amaurosis-10 (LCA10) in a subject in need thereof, the method involves contacting a cell in the subject with a base editor polypeptide containing (a) a nucleic acid programmable DNA binding protein (napDNAbp) domain and a deaminase domain, or (b) one or more polynucleotides encoding the base editor. The method further involves contacting the cell with one or more guide polynucleotides, or one or more polynucleotides encoding the one or more guide polynucleotides, where the guide polynucleotides target the base editor to effect an alteration of a nucleobase of a 290-KD centrosomal protein (CEP290) polynucleotide in the cell, thereby treating LCA10 in the subject.

In another aspect, the invention features a modified cell containing an alteration in a nucleobase of a CEP290 polynucleotide. The alteration increases expression and/or activity of the encoded CEP290 polypeptide as compared to a control cell without the alteration. The cell is prepared according to the method of any one of the above aspects, or embodiments thereof.

In another aspect, the invention features a base editor system containing two polynucleotides together encoding a base editor polypeptide. The base editor polypeptide contains a nucleic acid programmable DNA binding protein (napDNAbp) domain and a deaminase domain. A first polynucleotide encodes a fusion protein containing an N-terminal fragment of the base editor fused to a split intein-N. A second polynucleotide encodes a fusion protein containing the remaining C-terminal fragment the base editor fused to a split intein-C. The base editor system also contains one or more guide polynucleotides, or one or more polynucleotides encoding the one or more guide polynucleotides, where the one or more guide polynucleotides contain a spacer containing at least 10 contiguous nucleotides of a spacer corresponding to a nucleic acid sequence selected from one or more of:

(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

In another aspect, the invention features a base editor system containing one or more polynucleotides encoding a base editor polypeptide. The base editor polypeptide contains a nucleic acid programmable DNA binding protein (napDNAbp) domain and a deaminase domain. The base editor system also contains one or more guide polynucleotides, or one or more polynucleotides encoding the one or more guide polynucleotides, wherein the one or more guide polynucleotides, where the guide polynucleotides contain a spacer sequence containing at least 10 contiguous nucleotides of a spacer corresponding to a nucleic acid sequence selected from one or more of:

(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

In another aspect, the invention features a set of one or more polynucleotides encoding the base editor system of any aspect provided herein, or embodiments thereof, or a component thereof.

In another aspect, the invention features a vector containing a set of one or more polynucleotides of any aspect provided herein, or embodiments thereof.

In another aspect, the invention features a kit containing a base editor system containing a base editor polypeptide, or one or more polynucleotides encoding the same. The base editor polypeptide contains a nucleic acid programmable DNA binding protein (napDNAbp) domain and a deaminase domain. The base editor system also contains one or more guide polynucleotides containing, or one or more polynucleotides encoding the one or more guide polynucleotides, wherein the one or more guide polynucleotides contain a nucleic acid sequence selected from one or more of:

(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

In another aspect, the invention features a pharmaceutical composition containing an effective amount of a base editor system containing (a) a base editor polypeptide or one or (b) more polynucleotides encoding the base editor polypeptide. The base editor polypeptide contains a nucleic acid programmable DNA binding protein (napDNAbp) domain and a deaminase domain. The pharmaceutical composition also contains one or more guide polynucleotides, or one or more polynucleotides encoding the one or more guide polynucleotides, where the one or more guide polynucleotides contain a nucleic acid sequence selected from one or more of:

(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

In another aspect, the invention features a guide polynucleotide, or a polynucleotide encoding the guide polynucleotide, where the guide polynucleotide contains a nucleotide sequence selected from one or more of:

(SEQ ID NO: 438)
GAUACUCACAAUUACAACGUUUUAGAGCUAGAAAUAGCAAGUUAA
AAUAAGGCUAGUCCGUUAUCAACUUGAAAAAGUGGCACCGAGUCG
GUGCUUUUUU;
(SEQ ID NO: 439)
gGAUACUCACAAUUACAACGUUUUAGAGCUAGAAAUAGCAAGUUA
AAAUAAGGCUAGUCCGUUAUCAACUUGAAAAAGUGGCACCGAGUC
GGUGCUUUUUU;
(SEQ ID NO: 440)
GAGAUACUCACAAUUACAACGUUUUAGAGCUAGAAAUAGCAAGUU
AAAAUAAGGCUAGUCCGUUAUCAACUUGAAAAAGUGGCACCGAGU
CGGUGCUUUUUU;
(SEQ ID NO: 441)
gGAGAUACUCACAAUUACAACGUUUUAGAGCUAGAAAUAGCAAGU
UAAAAUAAGGCUAGUCCGUUAUCAACUUGAAAAAGUGGCACCGAG
UCGGUGCUUUUUU;
(SEQ ID NO: 442)
gUGAGAUACUCACAAUUACAACGUUUUAGAGCUAGAAAUAGCAAG
UUAAAAUAAGGCUAGUCCGUUAUCAACUUGAAAAAGUGGCACCGA
GUCGGUGCUUUUUU;
(SEQ ID NO: 443)
gAUGAGAUACUCACAAUUACAACGUUUUAGAGCUAGAAAUAGCAA
GUUAAAAUAAGGCUAGUCCGUUAUCAACUUGAAAAAGUGGCACCG
AGUCGGUGCUUUUUU;
(SEQ ID NO: 444)
GUAUCUCUGAUGAGUCCGUGAGGACGAAACGAGUAAGCUCGUCAG
AUACUCACAAUUACAACGUUUUAGAGCUAGAAAUAGCAAGUUAAA
AUAAGGCUAGUCCGUUAUCAACUUGAAAAAGUGGCACCGAGUCGG
UGCUUUUUU;
(SEQ ID NO: 445)
gAUCUCACUGAUGAGUCCGUGAGGACGAAACGAGUAAGCUCGUCU
GAGAUACUCACAAUUACAACGUUUUAGAGCUAGAAAUAGCAAGUU
AAAAUAAGGCUAGUCCGUUAUCAACUUGAAAAAGUGGCACCGAGU
CGGUGCUUUUUU;
(SEQ ID NO: 446)
gUAUCUCAUCUGAUGAGUCCGUGAGGACGAAACGAGUAAGCUCGU
CAUGAGAUACUCACAAUUACAACGUUUUAGAGCUAGAAAUAGCAA
GUUAAAAUAAGGCUAGUCCGUUAUCAACUUGAAAAAGUGGCACCG
AGUCGGUGCUUUUUU;
(SEQ ID NO: 447)
gCUCAUACUGAUGAGUCCGUGAGGACGAAACGAGUAAGCUCGUCU
AUGAGAUACUCACAAUUACAACGUUUUAGAGCUAGAAAUAGCAAG
UUAAAAUAAGGCUAGUCCGUUAUCAACUUGAAAAAGUGGCACCGA
GUCGGUGCUUUUUU;
(SEQ ID NO: 448)
gACUCACAAUUACAACUGGUUUUAGAGCUAGAAAUAGCAAGUUAA
AAUAAGGCUAGUCCGUUAUCAACUUGAAAAAGUGGCACCGAGUCG
GUGCUUUUUU;
(SEQ ID NO: 449)
gUACUCACAAUUACAACUGGUUUUAGAGCUAGAAAUAGCAAGUUA
AAAUAAGGCUAGUCCGUUAUCAACUUGAAAAAGUGGCACCGAGUC
GGUGCUUUUUU;
(SEQ ID NO: 450)
GAUACUCACAAUUACAACUGGUUUUAGAGCUAGAAAUAGCAAGUU
AAAAUAAGGCUAGUCCGUUAUCAACUUGAAAAAGUGGCACCGAGU
CGGUGCUUUUUU;
(SEQ ID NO: 451)
gGAUACUCACAAUUACAACUGGUUUUAGAGCUAGAAAUAGCAAGU
UAAAAUAAGGCUAGUCCGUUAUCAACUUGAAAAAGUGGCACCGAG
UCGGUGCUUUUUU;
(SEQ ID NO: 452)
GAGAUACUCACAAUUACAACUGGUUUUAGAGCUAGAAAUAGCAAG
UUAAAAUAAGGCUAGUCCGUUAUCAACUUGAAAAAGUGGCACCGA
GUCGGUGCUUUUUU;
(SEQ ID NO: 453)
gCAAUUACAACUGGGGCCGUUUUAGAGCUAGAAAUAGCAAGUUAA
AAUAAGGCUAGUCCGUUAUCAACUUGAAAAAGUGGCACCGAGUCG
GUGCUUUUUU;
(SEQ ID NO: 454)
gACAAUUACAACUGGGGCCGUUUUAGAGCUAGAAAUAGCAAGUUA
AAAUAAGGCUAGUCCGUUAUCAACUUGAAAAAGUGGCACCGAGUC
GGUGCUUUUUU;
(SEQ ID NO: 455)
gCACAAUUACAACUGGGGCCGUUUUAGAGCUAGAAAUAGCAAGUU
AAAAUAAGGCUAGUCCGUUAUCAACUUGAAAAAGUGGCACCGAGU
CGGUGCUUUUUU;
(SEQ ID NO: 456)
gUCACAAUUACAACUGGGGCCGUUUUAGAGCUAGAAAUAGCAAGU
UAAAAUAAGGCUAGUCCGUUAUCAACUUGAAAAAGUGGCACCGAG
UCGGUGCUUUUUU;
(SEQ ID NO: 457)
gCUCACAAUUACAACUGGGGCCGUUUUAGAGCUAGAAAUAGCAAG
UUAAAAUAAGGCUAGUCCGUUAUCAACUUGAAAAAGUGGCACCGA
GUCGGUGCUUUUUU;
(SEQ ID NO: 458)
gACUCACAAUUACAACUGGGGCCGUUUUAGAGCUAGAAAUAGCAA
GUUAAAAUAAGGCUAGUCCGUUAUCAACUUGAAAAAGUGGCACCG
AGUCGGUGCUUUUUU;
(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
(SEQ ID NO: 479)
andACUCACAAUUACAACUGGGGCC.

In any aspect provided herein, or embodiments thereof, the base editor effects a reversion of a pathogenic mutation to a non-pathogenic nucleotide. In any aspect provided herein, or embodiments thereof, the nucleobase is in an intron. In any aspect provided herein, or embodiments thereof, the alteration of the nucleobase disrupts a splice donor site. In embodiments, the splice donor site is a cryptic splice donor site. In any aspect provided herein, or embodiments thereof, the altered nucleobase in the CEP290 polynucleotide is associated with an alteration in splicing. In any aspect provided herein, or embodiments thereof, alteration of the nucleobase is associated with an increase in proper splicing of mRNA transcripts transcribed from the CEP290 polynucleotide. In any aspect provided herein, or embodiments thereof, the alteration is associated with an increase in levels of functional CEP290 polypeptides in the cell.

In any aspect provided herein, or embodiments thereof, the method further involves alleviating one or more symptoms of LCA10 in the subject. In any aspect provided herein, or embodiments thereof, the method further involves slowing or halting progression of vision loss associated with LCA10 in the subject. In any aspect provided herein, or embodiments thereof, the method further involves reducing loss of functional rod and/or cone cells associated with LCA10 in the subject.

In any aspect provided herein, or embodiments thereof, the base editor effects an alteration of a nucleobase selected from one or more of CEP290 c.2991+1651, CEP290 c.2991+1652, and CEP290 c.2991+1655. In any aspect provided herein, or embodiments thereof, the base editor effects a CEP290 c.2991+1655G>A alteration. In any aspect provided herein, or embodiments thereof, the base editor effects a CEP290 c.2991+1652T>C alteration. In any aspect provided herein, or embodiments thereof, the base editor effects a CEP290 c.2991+1652G>A alteration.

In any aspect provided herein, or embodiments thereof, the one or more guide polynucleotides contains a spacer containing from about 18 to about 23 nucleotides. In any aspect provided herein, or embodiments thereof, the one or more guide polynucleotides contains a spacer containing 19, 20, or 21 nucleotides. In any aspect provided herein, or embodiments thereof, the one or more guide polynucleotides contains a nucleic acid sequence containing at least 10 contiguous nucleotides of a spacer corresponding to a nucleic acid sequence selected from one or more of:

(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

In any aspect provided herein, or embodiments thereof, the one or more guide polynucleotides contains a sequence selected from one or more of:

(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGA
UACUCACAAUUACAAC;
(SEQ ID NO: 466)
AUCUCACUGAUGAGUCCGUGAGGACGAAACGAGUAAGCUCGUCUG
AGAUACUCACAAUUACAAC;
(SEQ ID NO: 467)
UAUCUCAUCUGAUGAGUCCGUGAGGACGAAACGAGUAAGCUCGUC
AUGAGAUACUCACAAUUACAAC;
(SEQ ID NO: 468)
CUCAUACUGAUGAGUCCGUGAGGACGAAACGAGUAAGCUCGUCUA
UGAGAUACUCACAAUUACAAC;
(SEQ ID NO: 469)
ACUCACAAUUACAACUG;
(SEQ ID NO: 4700)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

In any aspect provided herein, or embodiments thereof, the one or more guide polynucleotides contains a scaffold containing a nucleotide sequence with at least about 85% sequence identity to the following sequence:

(SEQ ID NO: 484)
GUUUUAGAGCUAGAAAUAGCAAGUUAAAAUAAGGCUAGUCCGUUAUCAA
CUUGAAAAAGUGGCACCGAGUCGGUGCUUUUUU.

In any aspect provided herein, or embodiments thereof, one or more guide polynucleotides contains a modified nucleotide. In any aspect provided herein, or embodiments thereof, the one or more guide polynucleotides contains one or more of a 2′-OMe and a phosphorothioate.

In any aspect provided herein, or embodiments thereof, the method involves (i) contacting the cell with a first polynucleotide encoding a fusion protein containing an N-terminal fragment of the base editor fused to a split intein-N, and (ii) contacting the cell with a second polynucleotide encoding a fusion protein containing the remaining C-terminal fragment of the base editor fused to a split intein-C. In any aspect provided herein, or embodiments thereof, the C-terminal amino acid of the N-terminal fragment of the base editor is positioned within the napDNAbp domain of the base editor. In any aspect provided herein, or embodiments thereof, the C-terminal amino acid of the N-terminal fragment of the base editor corresponds to position 573 of the napDNAbp and the N-terminal amino acid of the C-terminal fragment of the base editor corresponds to position 574 of the napDNAbp, wherein the napDNAbp amino acid position is referenced to the following sequence:

SpCas9
(SEQ ID NO: 197)
MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD.

In any aspect provided herein, or embodiments thereof, the split intein-N and split intein-C are components of a split intein selected from one or more of a Cfa, Cfa(GEP), Gp41.1, Gp41.8, IMPDH.1, NrdJ.1, and Npu. In any aspect provided herein, or embodiments thereof, the split intein-N and/or split intein-C contains an amino acid sequence selected from those corresponding to SEQ ID NOs: 371, 373, 375, 377, 390, 392, 394, 396, 398, 400, 401, 402, 490, 492, 494, 496, 498, 500, 502, 504, 506, 508, 509, and 510, or functional fragments thereof.

In any aspect provided herein, or embodiments thereof, the method further involves contacting the cell with a vector containing polynucleotide(s) encoding the base editor and/or the one or more guide polynucleotides. In any aspect provided herein, or embodiments thereof, the vector contains a lipid nanoparticle. In any aspect provided herein, or embodiments thereof, the vector is an adeno-associated virus (AAV) vector. In any aspect provided herein, or embodiments thereof, the AAV vector is an AAV5, PHB.EB, or PHP.B viral vector.

In any aspect provided herein, or embodiments thereof, the one or more polynucleotides encoding the base editor and/or one or more guide polynucleotides contain a promoter controlling expression of the base editor and/or one or more guide polynucleotides. In any aspect provided herein, or embodiments thereof, the promoter is selected from one or more of CMV, PR1.7, hG1.7, hGRK, and U6. In any aspect provided herein, or embodiments thereof, the promoter is selected from one or more of PR1.7, hG1.7, and hGRK. In any aspect provided herein, or embodiments thereof, the promoter facilitates expression of the base editor and/or one or more of the guide polynucleotides in a cone cell. In any aspect provided herein, or embodiments thereof, the promoter facilitates expression of the base editor and/or one or more of the guide polynucleotides in a rod cell.

In any aspect provided herein, or embodiments thereof, the napDNAbp domain contains a Cas9, Cas12a/Cpf1, Cas12b/C2c1, Cas12c/C2c3, Cas12d/CasY, Cas12e/CasX, Cas12g, Cas12h, Cas12i, or Cas12j/CasΦ polynucleotide or a functional portion thereof. In any aspect provided herein, or embodiments thereof, the napDNAbp domain contains a Cas9 polynucleotide or a functional portion thereof. In any aspect provided herein, or embodiments thereof, the napDNAbp domain contains a dead Cas9 (dCas9) or a Cas9 nickase (nCas9). In any aspect provided herein, or embodiments thereof, the napDNAbp domain contains a Staphylococcus aureus Cas9 (SaCas9), Streptococcus thermophilus 1 Cas9 (St1Cas9), a Streptococcus pyogenes Cas9 (SpCas9), or variants thereof. In any aspect provided herein, or embodiments thereof, the napDNAbp domain contains an SpCas9, or a variant thereof. In any aspect provided herein, or embodiments thereof, the napDNAbp domain contains a variant of SpCas9 having an altered protospacer-adjacent motif (PAM) specificity. In any aspect provided herein, or embodiments thereof, the SpCas9 variant recognizes a PAM sequence selected from the group consisting of NGA, NGCG, NNNRRT, NGCG, NGCN, NGTN, and NGC.

In any aspect provided herein, or embodiments thereof, the deaminase domain is an adenosine deaminase, a cytidine deaminase domain, or a cytidine adenosine deaminase domain. In any aspect provided herein, or embodiments thereof, the adenosine deaminase domain converts a target A⋅T to G⋅C in the CEP290 polynucleotide. In any aspect provided herein, or embodiments thereof, the cytidine deaminase domain converts a target C⋅G to T⋅A in the CEP290 polynucleotide. In any aspect provided herein, or embodiments thereof, the cytidine deaminase domain contains an APOBEC deaminase domain or a derivative thereof. In any aspect provided herein, or embodiments thereof, the APOBEC deaminase domain is selected from one or more of rAPOBEC, ppAPOBEC, RrA3F, AmAPOBEC1, and SsAPOBEC3B. In any aspect provided herein, or embodiments thereof, the APOBEC deaminase domain contains a ppAPOBEC cytidine deaminase domain. In any aspect provided herein, or embodiments thereof, the APOBEC deaminase domain contains a rAPOBEC cytidine deaminase domain. In any aspect provided herein, or embodiments thereof, the APOBEC deaminase domain contains a pRrA3F cytidine deaminase domain. In any aspect provided herein, or embodiments thereof, the APOBEC deaminase domain contains a AmAPOBEC cytidine deaminase domain. In any aspect provided herein, or embodiments thereof, the APOBEC deaminase domain contains a SsAPOBEC cytidine deaminase domain. In any aspect provided herein, or embodiments thereof, the adenosine deaminase domain is TadA deaminase domain. In any aspect provided herein, or embodiments thereof, the adenosine deaminase domain is a TadA*8 or TadA*9 variant. In any aspect provided herein, or embodiments thereof, the base editor is a cytidine adenosine base editor.

In any aspect provided herein, or embodiments thereof, the method further involves expressing one or more uracil glycosylase inhibitors (UGIs) in the cell. In any aspect provided herein, or embodiments thereof, the UGI is not fused to the base editor. In any aspect provided herein, or embodiments thereof, the UTI is fused to the base editor. In any aspect provided herein, or embodiments thereof, the base editor polypeptide further contains one or more uracil glycosylase inhibitors (UGIs). In any aspect provided herein, or embodiments thereof, the base editor polypeptide further contains two uracil glycosylase inhibitors (UGIs).

In any aspect provided herein, or embodiments thereof, the base editor polypeptide further contains one or more nuclear localization sequences (NLS).

In any aspect provided herein, or embodiments thereof, the subject is a mammal. In embodiments, the mammal is a primate. In embodiments, the primate is a human.

In any aspect provided herein, or embodiments thereof, the subject is less than 10 years old. In any aspect provided herein, or embodiments thereof, the subject is less than 1 year old.

In any aspect provided herein, or embodiments thereof, the cell is in vivo. In any aspect provided herein, or embodiments thereof, the cell is a mammalian cell. In any aspect provided herein, or embodiments thereof, the cell is a retinal cell. In any aspect provided herein, or embodiments thereof, the cell is a rod cell or a cone cell.

In any aspect provided herein, or embodiments thereof, the method further involves administering the base editor and/or one or more guide polynucleotides to the subject by subretinal injection or subfoveal injection. In embodiments, the subretinal injection or subfoveal injection results in the formation of a bleb. In embodiments, the bleb has an internal diameter of less than 6 mm. In embodiments, the bleb has an internal diameter of less than 1 mm, 2 mm, 3, mm, 4 mm, or 5 mm.

In any aspect provided herein, or embodiments thereof, expression and/or function is increased by at least 1, 2, 3, 4, 5, 6, 7, 8, 9, or 10-fold as compared to a control cell without the alteration. In any aspect provided herein, or embodiments thereof, the alteration is associated with an increase in the number of functional CEP290 polypeptides expressed from the CEP290 polypeptide.

Any aspect provided herein, or embodiments thereof, further involving or containing a polynucleotide encoding one or more uracil glycosylase inhibitors (UGIs). In any aspect provided herein, or embodiments thereof, the base editor polypeptide does not contain the UGI. In any aspect provided herein, or embodiments thereof, the base editor polypeptide contains the UGI.

In any aspect provided herein, or embodiments thereof, the vector is a viral vector. In any aspect provided herein, or embodiments thereof, the vector targets a cone cell and/or a rod cell.

In any aspect provided herein, or embodiments thereof, the kit further contains written instructions for the use of the kit in the treatment of Leber congenital amaurosis 10 (LCA10).

In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Alligator mississippiensis (American alligator) APOBEC1 (AmAPOBEC1) polypeptide” is meant a cytidine deaminase polypeptide with at least about 85% amino acid sequence identity to the exemplary AmAPOBEC1 polypeptide sequence provided below, or a fragment thereof having cytidine deaminase activity.

### Exemplary AmAPOBEC1 Polypeptide Sequence:

(SEQ ID NO: 425)
MADSSEKMRGQYISRDTFEKNYKPIDGTKEAHLLCEIKWGKYGKPWLHW
CQNQRMNIHAEDYFMNNIFKAKKHPVHCYVTWYLSWSPCADCASKIVKF
LEERPYLKLTIYVAQLYYHTEEENRKGLRLLRSKKVIIRVMDISDYNYC
WKVFVSNQNGNEDYWPLQFDPWVKENYSRLLDIFWESKCRSPNPW.

By “Alligator mississippiensis (American alligator) APOBEC1 (AmAPOBEC1) polynucleotide” is meant a nucleic acid molecule encoding an AmAPOBEC1 polypeptide, as well as the introns, exons, 3′ untranslated regions, 5′ untranslated regions, and regulatory sequences associated with its expression, or fragments thereof. In embodiments, an AmAPOBEC1 polynucleotide is the genomic sequence, cDNA, mRNA, or gene associated with and/or required for AmAPOBEC1 expression. An exemplary AmAPOBEC1 nucleotide sequence is provided below.

### Exemplary AmAPOBEC1 Polynucleotide Sequence:

(SEQ ID NO: 426)
ATGGCCGACAGCTCCGAGAAGATGAGGGGCCAGTACATCAGCCGCGACA
CCTTTGAGAAGAATTATAAGCCCATCGATGGCACAAAGGAGGCCCACCT
GCTGTGCGAGATCAAGTGGGGCAAGTACGGCAAGCCTTGGCTGCACTGG
TGTCAGAATCAGCGGATGAACATCCACGCCGAGGACTATTTCATGAACA
ATATCTTTAAGGCCAAGAAGCACCCTGTGCACTGCTACGTGACCTGGTA
TCTGTCTTGGAGCCCATGCGCCGATTGTGCCTCCAAGATCGTGAAGTTC
CTGGAGGAGCGGCCCTACCTGAAGCTGACCATCTATGTGGCCCAGCTGT
ACTATCACACAGAGGAGGAGAATAGGAAGGGCCTGCGGCTGCTGCGGAG
CAAGAAAGTGATCATCCGCGTGATGGACATCTCCGATTACAACTATTGC
TGGAAGGTGTTCGTGTCTAACCAGAATGGCAACGAGGACTACTGGCCAC
TGCAGTTTGATCCCTGGGTGAAGGAGAATTATTCTCGGCTGCTGGATAT
CTTCTGGGAGTCCAAGTGTAGATCTCCCAACCCTTGG.

By “Pongo pygmaeus (Orangutan) APOBEC (ppAPOBEC) polypeptide” is meant a cytidine deaminase polypeptide with at least about 85% amino acid sequence identity to the exemplary ppAPOBEC polypeptide sequence provided below, or a fragment thereof having cytidine deaminase activity.

Exemplary ppAPOBEC Polypeptide Sequence:

(SEQ ID NO: 427)
MTSEKGPSTGDPTLRRRIESWEFDVFYDPRELRKETCLLYEIKWGMSRK
IWRSSGKNTTNHVEVNFIKKFTSERRFHSSISCSITWFLSWSPCWECSQ
AIREFLSQHPGVTLVIYVARLFWHMDQRNRQGLRDLVNSGVTIQIMRAS
EYYHCWRNFVNYPPGDEAHWPQYPPLWMMLYALELHCIILSLPPCLKIS
RRWQNHLAFFRLHLQNCHYQTIPPHILLATGLIHPSVTWR.

By “Pongo pygmaeus (Orangutan) APOBEC (ppAPOBEC) polynucleotide” is meant a nucleic acid molecule encoding an ppAPOBEC polypeptide, as well as the introns, exons, 3′ untranslated regions, 5′ untranslated regions, and regulatory sequences associated with its expression, or fragments thereof. In embodiments, an ppAPOBEC polynucleotide is the genomic sequence, cDNA, mRNA, or gene associated with and/or required for ppAPOBEC expression. An exemplary ppAPOBEC nucleotide sequence is provided below.

Exemplary ppAPOBEC Polynucleotide Sequence:

(SEQ ID NO: 428)
ATGACCTCTGAGAAGGGCCCTAGCACAGGCGACCCCACCCTGCGGCGGA
GAATCGAGAGCTGGGAGTTCGACGTGTTCTACGACCCTAGAGAACTGAG
AAAGGAAACCTGCCTGCTGTACGAGATCAAGTGGGGCATGAGCAGAAAG
ATCTGGCGGAGCTCTGGCAAGAACACCACCAACCACGTGGAAGTGAATT
TCATCAAGAAGTTCACCAGCGAGAGAAGGTTCCACAGCAGCATCAGCTG
CAGCATCACCTGGTTCCTGAGCTGGTCCCCTTGCTGGGAATGCAGCCAG
GCCATCAGAGAGTTCCTGAGCCAACACCCCGGAGTGACACTGGTGATCT
ACGTGGCCAGACTGTTCTGGCACATGGACCAGAGAAACAGACAGGGCCT
GAGAGATCTGGTCAACAGCGGCGTGACTATCCAGATCATGCGGGCCAGC
GAGTACTACCACTGTTGGCGGAACTTCGTGAACTACCCCCCCGGCGATG
AGGCCCACTGGCCTCAGTACCCTCCTCTGTGGATGATGCTGTACGCCCT
GGAACTGCACTGCATCATCCTGTCTCTGCCTCCATGTCTGAAGATCTCT
AGAAGATGGCAGAACCACCTGGCCTTCTTCAGACTGCACCTGCAGAATT
GCCACTACCAGACCATCCCCCCCCACATCCTGCTGGCTACAGGCCTGAT
CCACCCTTCTGTGACCTGGAGA.

By “rat APOBEC (rAPOBEC) polypeptide” is meant a cytidine deaminase polypeptide with at least about 85% amino acid sequence identity to the exemplary rAPOBEC polypeptide sequence provided below, or a fragment thereof having cytidine deaminase activity.

Exemplary rAPOBEC Polypeptide Sequence:

(SEQ ID NO: 429)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK.

By “rat APOBEC (rAPOBEC) polynucleotide” is meant a nucleic acid molecule encoding an rAPOBEC polypeptide, as well as the introns, exons, 3′ untranslated regions, 5′ untranslated regions, and regulatory sequences associated with its expression, or fragments thereof. In embodiments, an rAPOBEC polynucleotide is the genomic sequence, cDNA, mRNA, or gene associated with and/or required for rAPOBEC expression. An exemplary rAPOBEC nucleotide sequence is provided below.

Exemplary rAPOBEC Polynucleotide Sequence:

(SEQ ID NO: 430)
ATGAGCAGCGAGACAGGCCCTGTGGCTGTGGATCCTACACTGCGGAGAA
GAATCGAGCCCCACGAGTTCGAGGTGTTCTTCGACCCCAGAGAGCTGCG
GAAAGAGACATGCCTGCTGTACGAGATCAACTGGGGCGGCAGACACTCT
ATCTGGCGGCACACAAGCCAGAACACCAACAAGCACGTGGAAGTGAACT
TTATCGAGAAGTTTACGACCGAGCGGTACTTCTGCCCCAACACCAGATG
CAGCATCACCTGGTTTCTGAGCTGGTCCCCTTGCGGCGAGTGCAGCAGA
GCCATCACCGAGTTTCTGTCCAGATATCCCCACGTGACCCTGTTCATCT
ATATCGCCCGGCTGTACCACCACGCCGATCCTAGAAATAGACAGGGACT
GCGCGACCTGATCAGCAGCGGAGTGACCATCCAGATCATGACCGAGCAA
GAGAGCGGCTACTGCTGGCGGAACTTCGTGAACTACAGCCCCAGCAACG
AAGCCCACTGGCCTAGATATCCTCACCTGTGGGTCCGACTGTACGTGCT
GGAACTGTACTGCATCATCCTGGGCCTGCCTCCATGCCTGAACATCCTG
AGAAGAAAGCAGCCTCAGCTGACCTTCTTCACAATCGCCCTGCAGAGCT
GCCACTACCAGAGACTGCCTCCACACATCCTGTGGGCCACCGGACTTAA
G.

By “Rhinopithecus roxellana (golden snub-nosed monkey) APOBEC3F (A3F) (RrA3F) polypeptide” is meant a cytidine deaminase polypeptide with at least about 85% amino acid sequence identity to the exemplary RrA3F polypeptide sequence provided below, or a fragment thereof having cytidine deaminase activity.

### Exemplary RrA3F Polypeptide Sequence:

(SEQ ID NO: 431)
MKPQIRDHRPNPMEAMYPHIFYFHFENLEKAYGRNETWLCFTVEIIKQY
LPVPWKKGVFRNQVDPETHCHAEKCFLSWFCNNTLSPKKNYQVTWYTSW
SPCPECAGEVAEFLAEHSNVKLTIYTARLYYFWDTDYQEGLRSLSEEGA
SVEIMDYEDFQYCWENFVYDDGEPFKRWKGLKYNFQSLTRRLREILQ.

By “Rhinopithecus roxellana (golden snub-nosed monkey) APOBEC3F (A3F) (RrA3F) polynucleotide” is meant a nucleic acid molecule encoding an RrA3F polypeptide, as well as the introns, exons, 3′ untranslated regions, 5′ untranslated regions, and regulatory sequences associated with its expression, or fragments thereof. In embodiments, an RrA3F polynucleotide is the genomic sequence, cDNA, mRNA, or gene associated with and/or required for RrA3F expression. An exemplary RrA3F nucleotide sequence is provided below.

### Exemplary RrA3F Polynucleotide Sequence:

(SEQ ID NO: 432)
ATGAAGCCCCAGATCAGGGACCACCGCCCCAATCCTATGGAGGCCATGT
ACCCTCACATCTTCTATTTTCACTTCGAGAACCTGGAGAAGGCCTACGG
CCGGAATGAGACCTGGCTGTGCTTTACAGTGGAGATCATCAAGCAGTAT
CTGCCAGTGCCCTGGAAGAAGGGCGTGTTCCGGAACCAGGTGGATCCAG
AGACCCACTGCCACGCCGAGAAGTGTTTTCTGTCCTGGTTCTGTAACAA
TACACTGTCTCCCAAGAAGAATTACCAGGTGACCTGGTATACAAGCTGG
TCCCCTTGCCCAGAGTGTGCAGGAGAGGTGGCAGAGTTTCTGGCAGAGC
ACAGCAACGTGAAGCTGACCATCTACACAGCCCGGCTGTACTATTTCTG
GGACACCGATTATCAGGAGGGCCTGAGATCTCTGAGCGAGGAGGGCGCC
TCCGTGGAGATCATGGACTACGAGGATTTTCAGTATTGCTGGGAGAACT
TCGTGTACGACGATGGCGAGCCTTTTAAGAGGTGGAAGGGCCTGAAGTA
TAATTTCCAGTCTCTGACACGGAGACTGCGCGAGATCCTGCAG.

By “Sus scrofa (pig) APOBEC3B (SsAPOBEC3B) polypeptide” is meant a cytidine deaminase polypeptide with at least about 85% amino acid sequence identity to the exemplary SsAPOBEC3B polypeptide sequence provided below, or a fragment thereof having cytidine deaminase activity.

### Exemplary SsAPOBEC3B Polypeptide Sequence:

(SEQ ID NO: 433)
MDPQRLRQWPGPGPASRGGYGQRPRIRNPEEWFHELSPRTFSFHFRNLR
FASGRNRSYICCQVEGKNCFFQGIFQNQVPPDPPCHAELCFLSWFQSWG
LSPDEHYYVTWFISWSPCCECAAKVAQFLEENRNVSLSLSAARLYYFWK
SESREGLRRLSDLGAQVGIMSFQDFQHCWNNFVHNLGMPFQPWKKLHKN
YQRLVTELKQILREEPATYGSPQAQGKVRIGSTAAGLRHSHSHTRSEAH
LRPNHSSRQHRILNPPREARARTCVLVDASWICYR.

By “Sus scrofa (pig) APOBEC3B (SsAPOBEC3B) polynucleotide” is meant a nucleic acid molecule encoding an SsAPOBEC3B polypeptide, as well as the introns, exons, 3′ untranslated regions, 5′ untranslated regions, and regulatory sequences associated with its expression, or fragments thereof. In embodiments, an SsAPOBEC3B polynucleotide is the genomic sequence, cDNA, mRNA, or gene associated with and/or required for SsAPOBEC3B expression. An exemplary SsAPOBEC3B nucleotide sequence is provided below.

### Exemplary SsAPOBEC3B Polynucleotide Sequence:

(SEQ ID NO: 434)
ATGGACCCACAGAGGCTGCGCCAGTGGCCCGGCCCTGGCCCAGCAAGCA
GGGGCGGCTACGGCCAGCGGCCAAGAATCAGGAACCCCGAGGAGTGGTT
TCACGAGCTGTCTCCCCGGACCTTCAGCTTTCACTTCCGCAACCTGAGG
TTCGCATCCGGCCGCAATCGGTCTTATATCTGCTGTCAGGTGGAGGGCA
AGAACTGCTTCTTTCAGGGCATCTTTCAGAATCAGGTGCCACCTGACCC
ACCATGCCACGCAGAGCTGTGCTTCCTGTCTTGGTTCCAGAGCTGGGGC
CTGTCCCCCGATGAGCACTACTATGTGACATGGTTTATCTCTTGGAGCC
CTTGCTGTGAGTGTGCCGCCAAGGTGGCCCAGTTCCTGGAGGAGAACCG
CAACGTGAGCCTGTCTCTGAGCGCCGCAAGGCTGTACTATTTCTGGAAG
TCCGAGTCTAGAGAGGGACTGCGGAGACTGAGCGACCTGGGAGCACAAG
TGGGAATCATGTCCTTTCAGGATTTCCAGCACTGCTGGAACAATTTTGT
GCACAACCTGGGCATGCCCTTCCAGCCTTGGAAGAAGCTGCACAAGAAT
TACCAGAGGCTGGTGACCGAGCTGAAGCAGATCCTGCGCGAGGAGCCTG
CCACATATGGCTCTCCACAGGCCCAGGGCAAGGTGAGAATCGGAAGCAC
CGCAGCAGGACTGAGGCACAGCCACTCCCACACACGCTCCGAGGCACAC
CTGAGGCCTAACCACAGCTCCAGACAGCACAGGATCCTGAATCCTCCAC
GGGAGGCCAGAGCCAGGACCTGCGTGCTGGTGGATGCCTCTTGGATCTG
TTACAGA.

By “adenine” or “9H-Purin-6-amine” is meant a purine nucleobase with the molecular formula C₅H₅N₅, having the structure

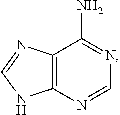

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

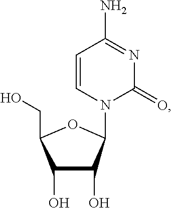

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence:

(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD,

or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route. In embodiments, one or more compositions described herein are administered by subretinal or subfoveal injection. In some instances, subretinal injection creates a bleb in the fovea.

By “agent” is meant any small molecule chemical compound, antibody, nucleic acid molecule, or polypeptide, or fragments thereof.

By “alteration” is meant a change in the level, structure, or activity of an analyte, gene or polypeptide as detected by standard art known methods such as those described herein. As used herein, an alteration includes a change (e.g., increase or decrease) in expression levels. In embodiments, the increase or decrease in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n(D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

By “bleb” is meant a small fluid-filled blister. In embodiments, a bleb has an internal diameter of no more than about 10 mm, 9 mm, 8 mm, 7 mm, 6 mm, 5 mm, 4 mm, 3 mm, 2 mm, or 1 mm. In embodiments, a bleb has a maximum internal height of less than about 2 mm, 1 mm, 0.5 mm, 0.25 mm, or 0.1 mm. In embodiments, the internal volume of a bleb is approximately equal to the volume of an amount of a composition of the present invention administered to a patient. In some cases, the internal volume of the bleb is about or at least about 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, or 500 μL. In some cases, the internal volume of the bleb is no more than about 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, or 500 μL.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease. By “290-KD centrosomal protein (CEP290) polypeptide” is meant a protein with at least about 85% amino acid sequence identity to Ensembl Accession No. ENST00000552810.6, or a fragment thereof that functions in ciliary assembly and/or ciliary trafficking. An exemplary CEP290 amino acid sequence from Homo sapiens is provided below (Ensembl: ENST00000552810.6; GenBank Accession no: DQ109808.1).

>ENST00000552810.6 peptide: ENSP00000448012 pep: protein_coding
(protein_id = ″AAZ83370.1″)
(SEQ ID NO: 435)
MPPNINWKEIMKVDPDDLPRQEELADNLLISLSKVEVNELKSEKQENVIHLFRITQSLMKMK
AQEVELALEEVEKAGEEQAKFENQLKTKVMKLENELEMAQQSAGGRDTRFLRNEICQLEKQL
EQKDRELEDMEKELEKEKKVNEQLALRNEEAENENSKLRRENKRLKKKNEQLCQDIIDYQKQ
IDSQKETLLSRRGEDSDYRSQLSKKNYELIQYLDEIQTLTEANEKIEVQNQEMRKNLEESVQ
EMEKMTDEYNRMKAIVHQTDNVIDQLKKENDHYQLQVQELTDLLKSKNEEDDPIMVAVNAKV
EEWKLILSSKDDEIIEYQQMLHNLREKLKNAQLDADKSNVMALQQGIQERDSQIKMLTEQVE
QYTKEMEKNTCIIEDLKNELQRNKGASTLSQQTHMKIQSTLDILKEKTKEAERTAELAEADA
REKDKELVEALKRLKDYESGVYGLEDAVVEIKNCKNQIKIRDREIEILTKEINKLELKISDF
LDENEALRERVGLEPKTMIDLTEFRNSKHLKQQQYRAENQILLKEIESLEEERLDLKKKIRQ
MAQERGKRSATSGLTTEDLNLTENISQGDRISERKLDLLSLKNMSEAQSKNEFLSRELIEKE
RDLERSRTVIAKFQNKLKELVEENKQLEEGMKEILQAIKEMQKDPDVKGGETSLIIPSLERL
VNAIESKNAEGIFDASLHLKAQVDQLTGRNEELRQELRESRKEAINYSQQLAKANLKIDHLE
KETSLLRQSEGSNVVFKGIDLPDGIAPSSASIINSQNEYLIHLLQELENKEKKLKNLEDSLE
DYNRKFAVIRHQQSLLYKEYLSEKETWKTESKTIKEEKRKLEDQVQQDAIKVKEYNNLLNAL
QMDSDEMKKILAENSRKITVLQVNEKSLIRQYTTLVELERQLRKENEKQKNELLSMEAEVCE
KIGCLQRFKEMAIFKIAALQKVVDNSVSLSELELANKQYNELTAKYRDILQKDNMLVQRTSN
LEHLECENISLKEQVESINKELEITKEKLHTIEQAWEQETKLGNESSMDKAKKSITNSDIVS
ISKKITMLEMKELNERQRAEHCQKMYEHLRTSLKQMEERNFELETKFAELTKINLDAQKVEQ
MLRDELADSVSKAVSDADRQRILELEKNEMELKVEVSKLREISDIARRQVEILNAQQQSRDK
EVESLRMQLLDYQAQSDEKSLIAKLHQHNVSLQLSEATALGKLESITSKLQKMEAYNLRLEQ
KLDEKEQALYYARLEGRNRAKHLRQTIQSLRRQFSGALPLAQQEKFSKTMIQLQNDKLKIMQ
EMKNSQQEHRNMENKTLEMELKLKGLEELISTLKDTKGAQKVINWHMKIEELRLQELKLNRE
LVKDKEEIKYLNNIISEYERTISSLEEEIVQQNKFHEERQMAWDQREVDLERQLDIFDRQQN
EILNAAQKFEEATGSIPDPSLPLPNQLEIALRKIKENIRIILETRATCKSLEEKLKEKESAL
RLAEQNILSRDKVINELRLRLPATAEREKLIAELGRKEMEPKSHHTLKIAHQTIANMQARLN
QKEEVLKKYQRLLEKAREEQREIVKKHEEDLHILHHRLELQADSSLNKFKQTAWDLMKQSPT
PVPTNKHFIRLAEMEQTVAEQDDSLSSLLVKLKKVSQDLERQREITELKVKEFENIKLQLQE
NHEDEVKKVKAEVEDLKYLLDQSQKESQCLKSELQAQKEANSRAPTTTMRNLVERLKSQLAL
KEKQQKALSRALLELRAEMTAAAEERIISATSQKEAHLNVQQIVDRHTRELKTQVEDLNENL
LKLKEALKTSKNRENSLTDNLNDLNNELQKKQKAYNKILREKEEIDQENDELKRQIKRLTSG
LQGKPLTDNKQSLIEELQRKVKKLENQLEGKVEEVDLKPMKEKNAKEELIRWEEGKKWQAKI
EGIRNKLKEKEGEVFTLTKQLNTLKDLFAKADKEKLTLQRKLKTTGMTVDQVLGIRALESEK
ELEELKKRNLDLENDILYMRAHQALPRDSVVEDLHLQNRYLQEKLHALEKQFSKDTYSKPSI
SGIESDDHCQREQELQKENLKLSSENIELKFQLEQANKDLPRLKNQVRDLKEMCEFLKKEKA
EVQRKLGHVRGSGRSGKTIPELEKTIGLMKKVVEKVQRENEQLKKASGILTSEKMANIEQEN
EKLKAELEKLKAHLGHQLSMHYESKTKGTEKIIAENERLRKELKKETDAAEKLRIAKNNLEI
LNEKMTVQLEETGKRLQFAESRGPQLEGADSKSWKSIVVTRMYETKLKELETDIAKKNQSIT
DLKQLVKEATEREQKVNKYNEDLEQQIKILKHVPEGAETEQGLKRELQVLRLANHQLDKEKA
ELIHQIEANKDQSGAESTIPDADQLKEKIKDLETQLKMSDLEKQHLKEEIKKLKKELENFDP
SFFEEIEDLKYNYKEEVKKNILLEEKVKKLSEQLGVELTSPVAASEEFEDEEESPVNFPIY.

By “290-KD centrosomal protein (CEP290) polynucleotide” is meant a nucleic acid molecule encoding an CEP290 polypeptide, as well as the introns, exons, 3′ untranslated regions, 5′ untranslated regions, and regulatory sequences associated with its expression, or fragments thereof. In embodiments, an CEP290 polynucleotide is the genomic sequence, cDNA, mRNA, or gene associated with and/or required for CEP290 expression. Exemplary CEP290 nucleotide sequences from Homo sapiens are provided below and at SEQ ID NO: 437 (corresponding to Ensembl Accession Nos.: ENST00000552810.6 and ENSG00000198707), where nucleotides in exons are in bold text, nucleotides in introns are in plain text, and the nucleotide c.2991+1655A is indicated by a bold double-underlined “A” (A). In the notation c.2991+1655A, “c.2991” indicates coding nucleotide number 2991 in a CEP290 polynucleotide sequence and “+1655A” indicates an adenosine at nucleotide position 1655 of the intron of the CEP290 polynucleotide sequence immediately following coding nucleotide number 2991.

>ENST00000552810.6 cds: protein_coding
(SEQ ID NO: 436)
ATGCCACCTAATATAAACTGGAAAGAAATAATGAAAGTTGACCCAGATGACCTGCCCCGTCA
AGAAGAACTGGCAGATAATTTATTGATTTCCTTATCCAAGGTGGAAGTAAATGAGCTAAAAA
GTGAAAAGCAAGAAAATGTGATACACCTTTTCAGAATTACTCAGTCACTAATGAAGATGAAA
GCTCAAGAAGTGGAGCTGGCTTTGGAAGAAGTAGAAAAAGCTGGAGAAGAACAAGCAAAATT
TGAAAATCAATTAAAAACTAAAGTAATGAAACTGGAAAATGAACTGGAGATGGCTCAGCAGT
CTGCAGGTGGACGAGATACTCGGTTTTTACGTAATGAAATTTGCCAACTTGAAAAACAATTA
GAACAAAAAGATAGAGAATTGGAGGACATGGAAAAGGAGTTGGAGAAAGAGAAGAAAGTTAA
TGAGCAATTGGCTCTTCGAAATGAGGAGGCAGAAAATGAAAACAGCAAATTAAGAAGAGAGA
ACAAACGTCTAAAGAAAAAGAATGAACAACTTTGTCAGGATATTATTGACTACCAGAAACAA
ATAGATTCACAGAAAGAAACACTTTTATCAAGAAGAGGGGAAGACAGTGACTACCGATCACA
GTTGTCTAAAAAAAACTATGAGCTTATCCAATATCTTGATGAAATTCAGACTTTAACAGAAG
CTAATGAGAAAATTGAAGTTCAGAATCAAGAAATGAGAAAAAATTTAGAAGAGTCTGTACAG
GAAATGGAGAAGATGACTGATGAATATAATAGAATGAAAGCTATTGTGCATCAGACAGATAA
TGTAATAGATCAGTTAAAAAAAGAAAACGATCATTATCAACTTCAAGTGCAGGAGCTTACAG
ATCTTCTGAAATCAAAAAATGAAGAAGATGATCCAATTATGGTAGCTGTCAATGCAAAAGTA
GAAGAATGGAAGCTAATTTTGTCTTCTAAAGATGATGAAATTATTGAGTATCAGCAAATGTT
ACATAACCTAAGGGAGAAACTTAAGAATGCTCAGCTTGATGCTGATAAAAGTAATGTTATGG
CTCTACAGCAGGGTATACAGGAACGAGACAGTCAAATTAAGATGCTCACCGAACAAGTAGAA
CAATATACAAAAGAAATGGAAAAGAATACTTGTATTATTGAAGATTTGAAAAATGAGCTCCA
AAGAAACAAAGGTGCTTCAACCCTTTCTCAACAGACTCATATGAAAATTCAGTCAACGTTAG
ACATTTTAAAAGAGAAAACTAAAGAGGCTGAGAGAACAGCTGAACTGGCTGAGGCTGATGCT
AGGGAAAAGGATAAAGAATTAGTTGAGGCTCTGAAGAGGTTAAAAGATTATGAATCGGGAGT
ATATGGTTTAGAAGATGCTGTCGTTGAAATAAAGAATTGTAAAAACCAAATTAAAATAAGAG
ATCGAGAGATTGAAATATTAACAAAGGAAATCAATAAACTTGAATTGAAGATCAGTGATTTC
CTTGATGAAAATGAGGCACTTAGAGAGCGTGTGGGCCTTGAACCAAAGACAATGATTGATTT
AACTGAATTTAGAAATAGCAAACACTTAAAACAGCAGCAGTACAGAGCTGAAAACCAGATTC
TTTTGAAAGAGATTGAAAGTCTAGAGGAAGAACGACTTGATCTGAAAAAAAAAATTCGTCAA
ATGGCTCAAGAAAGAGGAAAAAGAAGTGCAACTTCAGGATTAACCACTGAGGACCTGAACCT
AACTGAAAACATTTCTCAAGGAGATAGAATAAGTGAAAGAAAATTGGATTTATTGAGCCTCA
AAAATATGAGTGAAGCACAATCAAAGAATGAATTTCTTTCAAGAGAACTAATTGAAAAAGAA
AGAGATTTAGAAAGGAGTAGGACAGTGATAGCCAAATTTCAGAATAAATTAAAAGAATTAGT
TGAAGAAAATAAGCAACTTGAAGAAGGTATGAAAGAAATATTGCAAGCAATTAAGGAAATGC
AGAAAGATCCTGATGTTAAAGGAGGAGAAACATCTCTAATTATCCCTAGCCTTGAAAGACTA
GTTAATGCTATAGAATCAAAGAATGCAGAAGGAATCTTTGATGCGAGTCTGCATTTGAAAGC
CCAAGTTGATCAGCTTACCGGAAGAAATGAAGAATTAAGACAGGAGCTCAGGGAATCTCGGA
AAGAGGCTATAAATTATTCACAGCAGTTGGCAAAAGCTAATTTAAAGATAGACCATCTTGAA
AAAGAAACTAGTCTTTTACGACAATCAGAAGGATCAAATGTTGTTTTTAAAGGAATTGACTT
ACCTGATGGGATAGCACCATCTAGTGCCAGTATCATTAATTCTCAGAATGAATATTTAATAC
ATTTGTTACAGGAACTAGAAAATAAAGAAAAAAAGTTAAAGAATTTAGAAGATTCTCTTGAA
GATTACAACAGAAAATTTGCTGTAATTCGTCATCAACAAAGTTTGTTGTATAAAGAATACCT
AAGTGAAAAGGAGACCTGGAAAACAGAATCTAAAACAATAAAAGAGGAAAAGAGAAAACTTG
AGGATCAAGTCCAACAAGATGCTATAAAAGTAAAAGAATATAATAATTTGCTCAATGCTCTT
CAGATGGATTCGGATGAAATGAAAAAAATACTTGCAGAAAATAGTAGGAAAATTACTGTTTT
GCAAGTGAATGAAAAATCACTTATAAGGCAATATACAACCTTAGTAGAATTGGAGCGACAAC
TTAGAAAAGAAAATGAGAAGCAAAAGAATGAATTGTTGTCAATGGAGGCTGAAGTTTGTGAA
AAAATTGGGTGTTTGCAAAGATTTAAGGAAATGGCCATTTTCAAGATTGCAGCTCTCCAAAA
AGTTGTAGATAATAGTGTTTCTTTGTCTGAACTAGAACTGGCTAATAAACAGTACAATGAAC
TGACTGCTAAGTACAGGGACATCTTGCAAAAAGATAATATGCTTGTTCAAAGAACAAGTAAC
TTGGAACACCTGGAGTGTGAAAACATCTCCTTAAAAGAACAAGTGGAGTCTATAAATAAAGA
ACTGGAGATTACCAAGGAAAAACTTCACACTATTGAACAAGCCTGGGAACAGGAAACTAAAT
TAGGTAATGAATCTAGCATGGATAAGGCAAAGAAATCAATAACCAACAGTGACATTGTTTCC
ATTTCAAAAAAAATAACTATGCTGGAAATGAAGGAATTAAATGAAAGGCAGCGGGCTGAACA
TTGTCAAAAAATGTATGAACACTTACGGACTTCGTTAAAGCAAATGGAGGAACGTAATTTTG
AATTGGAAACCAAATTTGCTGAGCTTACCAAAATCAATTTGGATGCACAGAAGGTGGAACAG
ATGTTAAGAGATGAATTAGCTGATAGTGTGAGCAAGGCAGTAAGTGATGCTGATAGGCAACG
GATTCTAGAATTAGAGAAGAATGAAATGGAACTAAAAGTTGAAGTGTCAAAACTGAGAGAGA
TTTCTGATATTGCCAGAAGACAAGTTGAAATTTTGAATGCACAACAACAATCTAGGGACAAG
GAAGTAGAGTCCCTCAGAATGCAACTGCTAGACTATCAGGCACAGTCTGATGAAAAGTCGCT
CATTGCCAAGTTGCACCAACATAATGTCTCTCTTCAACTGAGTGAGGCTACTGCTCTTGGTA
AGTTGGAGTCAATTACATCTAAACTGCAGAAGATGGAGGCCTACAACTTGCGCTTAGAGCAG
AAACTTGATGAAAAAGAACAGGCTCTCTATTATGCTCGTTTGGAGGGAAGAAACAGAGCAAA
ACATCTGCGCCAAACAATTCAGTCTCTACGACGACAGTTTAGTGGAGCTTTACCCTTGGCAC
AACAGGAAAAGTTCTCCAAAACAATGATTCAACTACAAAATGACAAACTTAAGATAATGCAA
GAAATGAAAAATTCTCAACAAGAACATAGAAATATGGAGAACAAAACATTGGAGATGGAATT
AAAATTAAAGGGCCTGGAAGAGTTAATAAGCACTTTAAAGGATACCAAAGGAGCCCAAAAGG
TAATCAACTGGCATATGAAAATAGAAGAACTTCGTCTTCAAGAACTTAAACTAAATCGGGAA
TTAGTCAAGGATAAAGAAGAAATAAAATATTTGAATAACATAATTTCTGAATATGAACGTAC
AATCAGCAGTCTTGAAGAAGAAATTGTGCAACAGAACAAGTTTCATGAAGAAAGACAAATGG
CCTGGGATCAAAGAGAAGTTGACCTGGAACGCCAACTAGACATTTTTGACCGTCAGCAAAAT
GAAATACTAAATGCGGCACAAAAGTTTGAAGAAGCTACAGGATCAATCCCTGACCCTAGTTT
GCCCCTTCCAAATCAACTTGAGATCGCTCTAAGGAAAATTAAGGAGAACATTCGAATAATTC
TAGAAACACGGGCAACTTGCAAATCACTAGAAGAGAAACTAAAAGAGAAAGAATCTGCTTTA
AGGTTAGCAGAACAAAATATACTGTCAAGAGACAAAGTAATCAATGAACTGAGGCTTCGATT
GCCTGCCACTGCAGAAAGAGAAAAGCTCATAGCTGAGCTAGGCAGAAAAGAGATGGAACCAA
AATCTCACCACACATTGAAAATTGCTCATCAAACCATTGCAAACATGCAAGCAAGGTTAAAT
CAAAAAGAAGAAGTATTAAAGAAGTATCAACGTCTTCTAGAAAAAGCCAGAGAGGAGCAAAG
AGAAATTGTGAAGAAACATGAGGAAGACCTTCATATTCTTCATCACAGATTAGAACTACAGG
CTGATAGTTCACTAAATAAATTCAAACAAACGGCTTGGGATTTAATGAAACAGTCTCCCACT
CCAGTTCCTACCAACAAGCATTTTATTCGTCTGGCTGAGATGGAACAGACAGTAGCAGAACA
AGATGACTCTCTTTCCTCACTCTTGGTCAAACTAAAGAAAGTATCACAAGATTTGGAGAGAC
AAAGAGAAATCACTGAATTAAAAGTAAAAGAATTTGAAAATATCAAATTACAGCTTCAAGAA
AACCATGAAGATGAAGTGAAAAAAGTAAAAGCGGAAGTAGAGGATTTAAAGTATCTTCTGGA
CCAGTCACAAAAGGAGTCACAGTGTTTAAAATCTGAACTTCAGGCTCAAAAAGAAGCAAATT
CAAGAGCTCCAACAACTACAATGAGAAATCTAGTAGAACGGCTAAAGAGCCAATTAGCCTTG
AAGGAGAAACAACAGAAAGCACTTAGTCGGGCACTTTTAGAACTCCGGGCAGAAATGACAGC
AGCTGCTGAAGAACGTATTATTTCTGCAACTTCTCAAAAAGAGGCCCATCTCAATGTTCAAC
AAATCGTTGATCGACATACTAGAGAGCTAAAGACACAAGTTGAAGATTTAAATGAAAATCTT
TTAAAATTGAAAGAAGCACTTAAAACAAGTAAAAACAGAGAAAACTCACTAACTGATAATTT
GAATGACTTAAATAATGAACTGCAAAAGAAACAAAAAGCCTATAATAAAATACTTAGAGAGA
AAGAGGAAATTGATCAAGAGAATGATGAACTGAAAAGGCAAATTAAAAGACTAACCAGTGGA
TTACAGGGCAAACCCCTGACAGATAATAAACAAAGTCTAATTGAAGAACTCCAAAGGAAAGT
TAAAAAACTAGAGAACCAATTAGAGGGAAAGGTGGAGGAAGTAGACCTAAAACCTATGAAAG
AAAAGAATGCTAAAGAAGAATTAATTAGGTGGGAAGAAGGTAAAAAGTGGCAAGCCAAAATA
GAAGGAATTCGAAACAAGTTAAAAGAGAAAGAGGGGGAAGTCTTTACTTTAACAAAGCAGTT
GAATACTTTGAAGGATCTTTTTGCCAAAGCCGATAAAGAGAAACTTACTTTGCAGAGGAAAC
TAAAAACAACTGGCATGACTGTTGATCAGGTTTTGGGAATACGAGCTTTGGAGTCAGAAAAA
GAATTGGAAGAATTAAAAAAGAGAAATCTTGACTTAGAAAATGATATATTGTATATGAGGGC
CCACCAAGCTCTTCCTCGAGATTCTGTTGTAGAAGATTTACATTTACAAAATAGATACCTCC
AAGAAAAACTTCATGCTTTAGAAAAACAGTTTTCAAAGGATACATATTCTAAGCCTTCAATT
TCAGGAATAGAGTCAGATGATCATTGTCAGAGAGAACAGGAGCTTCAGAAGGAAAACTTGAA
GTTGTCATCTGAAAATATTGAACTGAAATTTCAGCTTGAACAAGCAAATAAAGATTTGCCAA
GATTAAAGAATCAAGTCAGAGATTTGAAGGAAATGTGTGAATTTCTTAAGAAAGAAAAAGCA
GAAGTTCAGCGGAAACTTGGCCATGTTAGAGGGTCTGGTAGAAGTGGAAAGACAATCCCAGA
ACTGGAAAAAACCATTGGTTTAATGAAAAAAGTAGTTGAAAAAGTCCAGAGAGAAAATGAAC
AGTTGAAAAAAGCATCAGGAATATTGACTAGTGAAAAAATGGCTAATATTGAGCAGGAAAAT
GAAAAATTGAAGGCTGAATTAGAAAAACTTAAAGCTCATCTTGGGCATCAGTTGAGCATGCA
CTATGAATCCAAGACCAAAGGCACAGAAAAAATTATTGCTGAAAATGAAAGGCTTCGTAAAG
AACTTAAAAAAGAAACTGATGCTGCAGAGAAATTACGGATAGCAAAGAATAATTTAGAGATA
TTAAATGAGAAGATGACAGTTCAACTAGAAGAGACTGGTAAGAGATTGCAGTTTGCAGAAAG
CAGAGGTCCACAGCTTGAAGGTGCTGACAGTAAGAGCTGGAAATCCATTGTGGTTACAAGAA
TGTATGAAACCAAGTTAAAAGAATTGGAAACTGATATTGCCAAAAAAAATCAAAGCATTACT
GACCTTAAACAGCTTGTAAAAGAAGCAACAGAGAGAGAACAAAAAGTTAACAAATACAATGA
AGACCTTGAACAACAGATTAAGATTCTTAAACATGTTCCTGAAGGTGCTGAGACAGAGCAAG
GCCTTAAACGGGAGCTTCAAGTTCTTAGATTAGCTAATCATCAGCTGGATAAAGAGAAAGCA
GAATTAATCCATCAGATAGAAGCTAACAAGGACCAAAGTGGAGCTGAAAGCACCATACCTGA
TGCTGATCAACTAAAGGAAAAAATAAAAGATCTAGAGACACAGCTCAAAATGTCAGATCTAG
AAAAGCAGCATTTGAAGGAGGAAATAAAGAAGCTGAAAAAAGAACTGGAAAATTTTGATCCT
TCATTTTTTGAAGAAATTGAAGATCTTAAGTATAATTACAAGGAAGAAGTGAAGAAGAATAT
TCTCTTAGAAGAGAAGGTAAAAAAACTTTCAGAACAATTGGGAGTTGAATTAACTAGCCCTG
TTGCTGCTTCTGAAGAGTTTGAAGATGAAGAAGAAAGTCCTGTTAATTTCCCCATTTACTAA.
>Portion of 12 dna: chromosome chromosome: GRCh38:12:88049016:88142088: −1
that contains the nucleotide c.2991 + 1655A (indicated by bold, double-
underline text)
(SEQ ID NO: 513)
GAAATGGCCATTTTCAAGATTGCAGCTCTCCAAAAAGTTGTAGATAATAGTGTTTCTTTGTC
TGAACTAGAACTGGCTAATAAACAGTACAATGAACTGACTGCTAAGTACAGGGACATCTTGC
AAAAAGATAATATGCTTGTTCAAAGAACAAGTAACTTGGAACACCTGGAGGTAAGTTTGTGT
GATTCTTGAACCTTGTGAAATTAGCCATTTTTCTTCAATATTTTTGTGTTTGGGGGGATTTG
GCAGATTTTAATTAAAGTTTGCCTGCATTTATATAAATTTAACAGAGATATAATTATCCATA
TTATTCATTCAGTTTAGTTATAAATATTTTGTTCCCACATAACACACACACACACACACAAT
ATATTATCTATTTATAGTGGCTGAATGACTTCTGAATGATTATCTAGATCATTCTCCTTAGG
TCACTTGCATGATTTAGCTGAATCAAACCTCTTTTAACCAGACATCTAAGAGAAAAAGGAGC
ATGAAACAGGTAGAATATTGTAATCAAAGGAGGGAAGCACTCATTAAGTGCCCATCCCTTTC
TCTTACCCCTGTACCCAGAACAAACTATTCTCCCATGGTCCCTGGCTTTTGTTCCTTGGAAT
GGATGTAGCCAACAGTAGCTGAAATATTAAGGGCTCTTCCTGGACCATGGATGCACTCTGTA
AATTCTCATCATTTTTTATTGTAGAATAAATGTAGAATTTTAATGTAGAATAAATTTATTTA
ATGTAGAATAAAAAATAAAAAAACTAGAGTAGAATATCATAAGTTACAATCTGTGAATATGG
ACCAGACCCTTTGTAGTTATCTTACAGCCACTTGAACTCTATACCTTTTACTGAGGACAGAA
CAAGCTCCTGATTTGTTCATCTTCCTCATCAGAAATAGAGGCTTATGGATTTTGGATTATTC
TTATCTAAGATCCTTTCACAGGAGTAGAATAAGATCTAATTCTATTAGCTCAAAAGCTTTTG
CTGGCTCATAGAGACACATTCAGTAAATGAAAACGTTGTTCTGAGTAGCTTTCAGGATTCCT
ACTAAATTATGAGTCATGTTTATCAATATTATTTAGAAGTAATCATAATCAGTTTGCTTTCT
GCTGCTTTTGCCAAAGAGAGGTGATTATGTTACTTTTTATAGAAAATTATGCCTATTTAGTG
TGGTGATAATTTATTTTTTTCCATTCTCCATGTCCTCTGTCCTATCCTCTCCAGCATTAGAA
AGTCCTAGGCAAGAGACATCTTGTGGATAATGTATCAATGAGTGATGTTTAACGTTATCATT
TTCCCAAAGAGTATTTTTCATCTTTCCTAAAGATTTTTTTTTTTTTTTTTTGAGATGGAGTT
TCATTCTGTCACCCAGGCTGAGTGCAGTGGCACGATCTCGGCTTAACGCTTACTGCATCCTC
TGCCTCCCAGATTCAAGCAGTTCTCCTGCCTCAGCCTCTGAGTAGCTGGGATTACAGGTGTG
CACCACCACACCAGCTAATTTTTTTTTTTTTTTTTTTTTTTTTGAGGCAGAGTCTCGCTCTG
TCACCCAGGCTGGAGTGCAGTGGCGCCATCTTGGCTCACTGCAAGCTCCACCTCCCGGGTTC
AGGCCGTTCTCCTGCCTCAGCCTCCTGAGTAGCTGGTACCACAGGCACCCACCATCATGCCC
GGCTAATTTTTTGTATTTTTAGTAGAGATGGGGTTTCACCTTGTTAGCCAGGATGGTGTCGA
TCTCCTGAACTCGTGATCCACCCGCCTCGGCCTCCTAAAGTGCTGGGATTACAGATGTGAGC
CACCGCACCTGGCCCCAGTTGTAATTGTGAATATCTCATACCTATCCCTATTGGCAGTGTCT
TAGTTTTATTTTTTATTATCTTTATTGTGGCAGCCATTATTCCTGTCTCTATCTCCAGTCTT
ACATCCTCCTTACTGCCACAAGAATGATCATTCTAAACATGAATCCTACCCTGTGACTCCCA
TGTGACTCCCCGCCTTAAAAACTGTCAAAAGCTACCGGTTACCTGAAGGGTAAAAGTCAAGT
CCCCTACTTACCTCATGTCATCTAGAGCAAGAGATGAACTAGCTGAGTTTTCTGACCACAGT
GTTCTTTCTTATGTATGTTCTTTTGTACGTGCTCTTTTCTATATATAGGGAACCATTTCTCT
CTTCCAGTTGTTTTGCTCAGTGAATTTCTATTCCTGTTTCAAAACTTGTTCAGGCATTACCT
TTTTTTTCTTAAGCATACTTTTTTTAATGGAACAAAGTCACTCCTGTCTACACTAGTTCTGC
ATCTTATACATAGGTTTTGTACATAGTACATATTTATATCACATCAAATTATATGTGTTTAC
ATATCTGTCTTCCTTAATGGAATATAAGTCTTTTGATATAAGGAACTATTTAATTTGTTTCT
GTGTGTTGAGTATCTCCTGTTTGGCACAGAGTTCAAGCTAATACATGAGAGTGATTAGTGGT
GGAGAGCCACAGTGCATGTGGTGTCAAATATGGTGCTTAGGAAATTATTGTTGCTTTTTGAG
AGGTAAAGGTTCATGAGACTAGAGGTCACGAAAATCAGATTTCATGTGTGAAGAATGGAATA
GATAATAAGGAAATACAAAAACTGGATGGGTAATAAAGCAAAAGAAAAACTTGAAATTTGAT
AGTAGAAGAAAAAAGAAATAGATGTAGATTGAGGTAGAATCAAGAAGAGGATTCTTTTTTTG
TTGTTTTTTTTTTTGAAACAGAGTCTCACTGTGTTGCCCAGGCTGGAGTGCAGTGGAGTGAT
CTTGGCTTACTGCAACCTCTGCCTCCCAGGTTCAAGCGATTCTTCTGCTTCAGTCTCCCGAG
TAGCTGGAATTACAGGTGCCCACCAGCACGGCCGGCTAATTTAGTAGAGACAGGGTTTTGCC
ATGTTGGCCGGGCTGGTCTCAAACTTTGGATCTCAGGTAATCCGCCAGCCTCAACTTCCCAA
AGTGCTGGGATTACAGGCATGAGCCACTGTGCCCAGCCTGTTTTTTTTTTTTTAAAGGAGAC
CAGTGAAGTTTCAGGAGGAGGGAAAGAAAATTTAGAGTTACTAGGGAGAGAGTGATGAAGAT
AAGAGATGAAAGTGGTAATAAGGGAAATAGCAAAATATCAGGGTAGGTGGGAGAAAAAGAGA
TTTGTAACAAACAATAGGATTATCCTGTGAAAAAGGATGAAAGGAAGAAAAAAATGGATAGA
AAGATATTTAAAACACCCTCAGCCTCCTGTTTTCCCTCCTGTGTATTCATAGTATATAAAAC
TATAATTATGTACTTTACTTAAAAAATATATTATTATTACCTTATCGTGCTTATTTAATCAT
AGCATGTCCTCTTTTTAGTCTCATTACCCTGTTTGTATTATTCTTCATAACACTTAATACCT
GACATTGTATTATATATTGGCTTATTTTCCAGGTACTCCACTCAAATATAAGTTCTAGGATA
TAATTTATTTATCACTGAAATCCATTGCTTAGAGTACCTGGCATGTAGTAAATAGGCATTCT
GTTTTTTCAAATAAAAAATAAAGGAACTTAAGATATATATTTATGTTATATCGCCAGCCTTT
TTCCTCACAGCTCTATTCTGTTGTACAGAATTACCTACTTTACAATTCCTGTGTTTCAAGGG
GATCTCAAATTTAACGTGTCCACAATGAACTCCTGATTTCTGTTTCTCTCCTAGTCATTCTT
ATTTCAATATATGTTCAGTTACCTAACCAGCTAGTCAAGGCAGATACTTTAGAGTTATTCTG
TAGTCATTCTTTTTCCCTACCATTTTTGTTTTCCAAATGTAATTTATGTGTGTCTTCTTCAT
CCTCGCAGCTCTAACCCTTGTCCAAACCAGCATCATCACTCATCTGGAGTTCCACAATGTCT
TTCTGGCTAGTTTCCCTGATTTCTCTATTGACCCCTTTATTCTCCACAGTGCAGCCAGAATG
ATTGTTTAAAACTTCCTCCTTAAAATCTTTAAATTGTTTTCTTTTATACGTTAAGTTAAATT
CCAGTTCCTTGTCTTGGCATGCCATGCCCTGCCTGGTGTGGCCCCTGATGGTCTCTCCAACT
TCATGTTTTACTACTATTGACTCTTATTTTTGCTTACTCTGCTTGGGTGCTCCAGTCCTCCA
AATCATTTCCTGCTCCAATCATTTCAATCATTTTTTCCTCTCAGATCTTATAGTATTCCAAA
TGCTTTCTTCCTTTGGAGCATCTGGGTTTACTAATAAATACTTCGTACCTCACAGTTCAGCT
TAAATATCAATTATTTGGTGGTTAAGACATCCTTCAACCGCTCTATCTAAATGTTCCTTTCT
ATTATTCACTGGCTCAGTACTCTGTTTTTATTTTCTTTCTAAATGTCAACTTTTTTTTTTTT
GAGTCAGGGTCTCACTGTTGCCCAGGCTCGAGTGCAGTTGCACAATCATAGCTCATTGCAGC
CTTGCCCTCCTGGGATCAAGTAATTCTCCCACCTCAGCCTCCAAAATAGCTGGGATTACAGG
TATGCATCACCATGCTCAGCTAATTTTTTGTGTTTTTTTGTAGAGATGAGGTCTCACTTTGT
TGCCCAGGCTGGTCTCAAACTCCTGGACTCAAGTGATTCTCCCACCTCAGCCTCCCAAAGTG
CTGGGGTTACAGGTGTGAGCCACTGCACCTGGTCGATACTGACTTTTTTTTTTTTTTGAGAT
GGAGTTTTGCTCTGTTGCCCAGGCTAGAGCGCAGTGGTGTGATCTCAGCTCACTGCAACCTC
CACCTCCCAGGTTAAAGGGATTCTTCTGCCTCAGTCTCCTGAGTAGCTGGGATTACAGGCAA
GTGCCATCATGACTGGCTAATTTTTGTATTTTTAGCACTATGTTTAGTACTGTGTTGGCCAG
GCTTGTCTCGAACTCCTGACCTCAAGTGATCCACCCACCTCAGCCTCCCAAAGTGCTGGGAT
TACAGGTGTGAGCCACCGTAATCGGCCAACATTGACATTTTTAGTAGACTTTTTGTTTGTTT
ACTTGCTTATTATCTGCTGCCTTCCACACTCTGGCGAAATCCTGCCACCCACCCACACACAC
ATAGGCACTGAATGGGCAGAACTCTGAAGGCCAGAATTTTATATTTCTTTTCACTGTAAACA
TCATCATCTGTCACTGATGGCACACTAGGATGCTCAGCAACTGTGTGCATGAAGGAAGTAAG
CACTAGTTTGTGAAGGCTGCAAAACTCTTGAGTATTCTAAGAGTTTTGGCCAAAATGAATGT
ACAGCTTTAGTGGCAGAAGCTAATACTCAGAAATTGAGGCCGTATATTGGATAACACAGGAT
TTGGATGATTATTTTAAAATAATATTTTACATTGTATATATGTGTGTGTGTGTGTGTGTGTG
TGTGTGTATGTGTGTGTGTGTGTATATATATATGTATGTATGTGTATTAGTCCGTTCTCATG
CTGCTATGAAGAAATACCTGAGACTGGGTAATTTATAAAGGAAAGAGGTTTAATTGACTCAC
AGTTCCACAGAGCTGGGGAGGCCTCAGAAAACTTAACAGTTATGGCAGAAGGGGAAGCAAAC
ACATTTTTCTTCACATGGTGGCCGGAATTAGAAGAATGTGAGCCGAGCAAAGGGGAAAGCCC
CTTATAAAACCATCAGACATCGTGAGAACTTACTATTATGAGAATAGCGTGGGGGAAACCAC
CCCCACGATTCAATTACCTCCCACCAAATCCCTCCCATGACATATGAGGATTATGGGAACTA
TGATTCAAGATGAGATTTGGGTAGGGACACAGCCAAACCATATCAGTATGTATATGTATACA
AGTATTATATATATATGTATGTGTTTGTATGCATACATGTATTATATATGGAGGAAATTCTA
ATTTTGTAAAAAACTGGATTGTGAGTTTTAAGGAGATGTTATATAAAGTTAAGACAATGTCA
TTTTGTGGTATTGGTCTGAATTACAATGTAGTTTCTTAGTGATATTTTTCCTTTATTCAGTG
TGAAAACATCTCCTTAAAAGAACAAGTGGAGTCTATAAATAAAGAACTGGAGATTACCAAGG
AAAAACTTCACACTATTGAACAAGCCTGGGAACAGGAAACTAAATTAG.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, 30 Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and 7-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

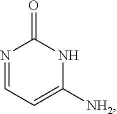

and corresponding to CAS No. 71-30-7.

By “cytidine” is meant a cytosine molecule attached to a ribose sugar via a glycosidic bond, having the structure

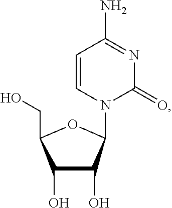

and corresponding to CAS No. 65-46-3.

Its molecular formula is C9H13N305.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence, or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include, but are not limited to, retinal dystrophies (e.g., severe retinal dystrophy), degenerative eye diseases, and congenital eye diseases (e.g., Leber's Congenital Amaurosis-10 (LCA10)). Without wishing to be bound by any particular theory, it has been recognized that some skilled artisans consider LCA to be a severe form of retinitis pigmentosa. Accordingly, the disclosure contemplates methods of treating severe forms retinitis pigmentosa.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent or active compound, e.g., a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue, or organ, but only to alter the pathogenic gene in about 1%, 5%, 10%, 25%, 50%, 75% or more of the cells present in a subject, tissue, or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment. By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In embodiments, the disease is a retinal dystrophy (e.g., severe retinal dystrophy) or Leber's Congenital Amaurosis-10 (LCA10). In some instances, the marker is a single nucleotide polymorphism, such as the IVS26 pathogenic mutation to the CEP290 gene (i.e., CEP290 c.2991+1655A>G). Non-limiting examples of markers a CEP290 transcript (e.g., a properly or improperly spliced transcript) or a CEP290 polypeptide (e.g., a full length or truncated CEP290 polypeptide).

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
(SEQ ID NO: 196)
MDSLLMNRRKFLYQFKNVRWAKGRRETYLC,
(SEQ ID NO: 328)
PKKKRKVEGADKRTADGSEFESPKKKRKV,
or
(SEQ ID NO: 329)
RKSGKIAAIVVKRPRKPKKKRKV.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasD, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-245, 254-260, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.). In some cases, the pathogenic mutation is the IVS26 mutation to the CEP290 gene, namely CEP290 c.2991+1655A>G.

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. In some cases, a reference is an untreated subject or cell. In some instances, a reference is a healthy cell that does not contain the IVS26 pathogenic mutation in the CEP290 gene (CEP290 c.2991+1655A>G). In some cases, a reference is a cell that does contain the IVS26 pathogenic mutation in the CEP290 gene (CEP290 c.2991+1655A>G). In embodiments, a reference is a healthy and/or untreated eye cell, retinal cell, cone cell, and/or rod cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9) or derivatives thereof (e.g., a sequence with at least about 85% sequence identity to a Cas9, such as Nme2Cas9 or spCas9).

The term “single nucleotide polymorphism (SNP)” is a variation in a single nucleotide that occurs at a specific position in the genome, where each variation is present to some degree within a population. In embodiments, an SNP is present in about or at least about 1, 2, 3, 4, or 5 individuals per 100,000 individuals in a population. SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration. A non-limiting example of an SNP is the IVS26 pathogenic mutation in the CEP290 gene (CEP290 c.2991+1655A>G).

By “specifically binds” is meant a nucleic acid molecule, polypeptide, polypeptide/polynucleotide complex, compound, or molecule that recognizes and binds a polypeptide and/or nucleic acid molecule of the invention, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the invention include any nucleic acid molecule that encodes a polypeptide of the invention or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition, or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>sp|P14739|UNGI_BPPB2 Uracil-DNA glycosylase inhibitor
(SEQ ID NO: 231)
MTNLSDIIEKETGKQLVIQESILMLPEEVEEVIGNKPESDILVHTAYDESTDENVMLLTSDA
PEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes. “Expression vectors” are nucleic acid sequences comprising the nucleotide sequence to be expressed in the recipient cell. Expression vectors contain a polynucleotide sequence as well as additional nucleic acid sequences to promote and/or facilitate the expression of the introduced sequence, such as start, stop, enhancer, promoter, and secretion sequences, into the genome of a mammalian cell. Examples of vectors include nucleic acid vectors, e.g., DNA vectors, such as plasmids, RNA vectors, viruses, or other suitable replicons (e.g., viral vectors). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Certain vectors that can be used for the expression of base editor polypeptides, base editor systems, or components thereof of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin. In embodiments, the vector is an AAV vector (e.g., AAV5, PHB.EB, or PHB.EB).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A and 1B provide schematics showing an overview of a mutation to CEP290 associated with Leber's Congenital Amaurosis-10. FIG. 1A provides a schematic showing splicing of a healthy version of the CEP290 gene. FIG. 1B provides a schematic showing splicing of a CEP290 gene containing a pathogenic c.2991+1655A>G mutation creating a new splice donor site (i.e., a cryptic splice donor site). The pathogenic mutation leads to a premature stop codon (e.g., p.Cys998X).

FIGS. 2A and 2B provide schematics showing base editing strategies for treating LCA-10. FIG. 2A provides a schematic showing a base editing strategy for direct correction of a pathogenic allele associated with LCA10. The c.2991+1655A>G mutation can be targeted using a cytidine base editor (CBE), where altering a C target base to a T results in reversion of the complementary (i.e., on the antisense strand) pathogenic G to an A. In FIG. 2A the bold “GT” indicates the splice donor site. The nucleotide sequences shown in FIG. 2A, correspond to SEQ ID NOs: 480 and 512, in order of occurrence. FIG. 2B provides a schematic showing a base editing strategy for disruption of a cryptic splice donor site associated with LCA10. The splice donor for the 128 bp cryptic exon associated with LCA10 is four bases upstream of the pathogenic c.2991+1655A>G allele mutation. The pathogenic A>G mutation makes the surrounding sequence a more ideal splice donor. The lower portion of FIG. 2B provides a schematic (Ma S L, et al. “Whole Exome Sequencing Reveals Novel PHEX Splice Site Mutations in Patients with Hypophosphatemic Rickets,” PLoS One. 2015 Jun. 24; 10(6):e0130729. doi: 10.1371/journal.pone.0130729. PMID: 26107949; PMCID: PMC4479593, the disclosure of which is incorporated herein by reference in its entirety for all purposes) showing a consensus sequence corresponding to splice donor sites across the human genome, where taller letters correspond to bases more frequently observed at a site and where the table below the letters provides a breakdown of the frequency with which each base (A, T, G, C) is observed at each site. In the schematic in the lower portion of FIG. 2B, “5′-Exon” indicates the end of an exon and +1 indicates the first base pair of the following exon 3′ of the Exon. The consensus splice donor “GT” (positions +1 and +2) upstream of the pathogenic mutation (position +5) can be disrupted using an adenosine deaminase (e.g., T>C alteration through deamination of the A complementary to the T) or using a cytidine deaminase (e.g., G>A alteration through deamination of the C complementary to the G).

FIGS. 3A and 3B provide schematics showing how base editing can be used to treat Leber's Congenital Amaurosis-10 (LCA10). FIG. 3A provides a schematic showing a target site containing a target base that can be altered by a cytidine deaminase base editor to treat LCA10. The sites labeled 9 and 11 in FIG. 3A indicate additional nucleotides in proximity to the target base that, in some embodiments, are altered along with or instead of the target base to result in one or more bystander edits (i.e., alterations of non-target bases). The cytidine deaminase base editor can deaminate the target cytidine (C) to result in a reversion of the complementary guanine (G) to an adenine (A). The nucleotide sequence shown in FIG. 3A (CCCAGTTGTAATTGTGAGTATCTCATA; SEQ ID NO: 480), corresponds to SEQ ID NO: 480. FIG. 3B shows a ˜300 bp lenti-integrated region of interest (ROI) that was incorporated into the genome of a HEK293T cell. The HEK293T cells containing the ROI were suitable for use in undertaking experiments to evaluate the use of base editor systems to treat LCA10. FIG. 3B shows how the unaltered ROI results in aberrant splicing of the gene into which it was integrated. By altering the target base shown in FIG. 3A within the region of interest of FIG. 3B proper splicing of the gene can be restored. To base edit the garget base in the HEK293T cells containing the ROI, the cells were lipofected with plasmid DNA encoding a cytidine deaminase base editor and a guide polynucleotide. Editing at the target site (FIG. 3A) was measured 5 days post-transfection.

FIG. 4 provides a bar graph showing percent (%) C-to-T conversion relative to the following target site: ATAC₇TC₉AC₁₁AATTACAACTGG (SEQ ID NO: 481) in a HEK293T cell, where the subscripts 7, 9, and 11 indicate the locations of the three base edits referenced in FIG. 4, namely, C7T, C9T, and C11T, respectively. The target site was edited using a plasmid-based rat APOBEC (rAPOBEC) BE4 editor driven by a CMV promoter and the guides indicated on the x-axis of FIG. 4 (see Table 1 for the guide sequences). The guides contained spacers with lengths varying between 18 and 23 nucleotides, and some of the guides included a self-cleaving hammerhead ribozyme (HRz). In FIG. 4, a guide targeting green fluorescent protein (GFP) was used as a control. In FIG. 4, “HRz” indicates a guide RNA containing a hammerhead ribozyme (HRz) at the 5′ end. In FIG. 4 each set of three bars corresponds, in order from left-to-right, to C7T, C9T, and C11T C>T conversion.

FIG. 5 provides a bar graph showing percent (%) C-to-T conversion relative to the following target site: ATAC₇TC₉AC₁₁AATTACAACTGG (SEQ ID NO: 481) in a HEK293T cell, where the subscripts 7, 9, and 11 indicate the locations of the three base edits referenced in FIG. 5, namely, C7T, C9T, and C11T, respectively. The target site was edited using a plasmid-based rat APOBEC (rAPOBEC) BE4 editor driven by a CMV promoter and the guides indicated on the x-axis of FIG. 5 (see Table 1 for the guide sequences). The guides contained spacers with lengths varying between 18 and 23 nucleotides. In FIG. 5, a guide targeting green fluorescent protein (GFP) was used as a control. In FIG. 5 each set of three bars corresponds, in order from left-to-right, to C7T, C9T, and C11T C>T conversion.

FIG. 6 provides a bar graph showing percent (%) C-to-T conversion relative to the following target site: ATAC₇TC₉AC₁₁AATTACAACTGG (SEQ ID NO: 481) in a HEK293T cell, where the subscripts 7, 9, and 11 indicate the locations of the three base edits referenced in FIG. 6, namely, C7T, C9T, and C11T, respectively. The target site was edited using split cytidine deaminase base editors (CBEs; see x-axis of FIG. 6) prepared using a Cfa(GEP) split intein fusion, where the editor was split at the amino acid residues corresponding to Glu573 and Cys574 of Cas9. The split editor was encoded by two separate plasmids. Each CBE fragment was expressed from a CMV promoter and a guide RNA was encoded in tandem on the plasmid that encoded the C-terminal split of the base editor and expressed under the control of a U6 promoter. The guides and base editors corresponding to each set of 3 bars in FIG. 6 are indicated on the x-axis, where sequences of the guide polynucleotides are provided in Table 1, and where “rAPOBEC” indicates a base editor containing “rat APOBEC,” “ppAPOBEC” indicates a base editor containing “Pongo pygmaeus (Orangutan) APOBEC,” RrA3F indicates a base editor containing “Rhinopithecus roxellana (golden snub-nosed monkey) APOBEC3F (A3F),” “AmAPOBEC1” indicates a base editor containing “Alligator mississippiensis (American alligator) APOBEC1,” and SsAPOBEC3B indicates a base editor containing “Sus scrofa (pig) APOBEC3B.” The guides contained spacers with lengths varying between 19 and 21 nucleotides. In FIG. 6 each set of three bars corresponds, in order from left-to-right, to C7T, C9T, and C11T C>T conversion.

FIG. 7 provides a bar graph showing percent (%) C-to-T conversion relative to the following target site: AC₅TC₇AC₉AATTACAACTGGGG (SEQ ID NO: 482) in a HEK293T cells, where the subscripts 5, 7, and 9 indicate the locations of the three base edits reference in FIG. 7, namely, C5T, C7T, and C9T. The target site was edited using a cytidine deaminase base editor (BE4 with rAPOBEC deaminase domain) in combination with the guides indicated on the x-axis, which contained spacers ranging in length from 18 to 22 nucleotides (see Table 1). In FIG. 7 each set of three bars corresponds, in order from left-to-right, to C5T, C7T, and C9T C>T conversion.

FIG. 8 provides a bar graph showing percent (%) C-to-T conversion at the following target site: CTC₂AC₄AATTAC₁₀AAC₁₃TGGGGCC (SEQ ID NO: 483) in a HEK293T cells, where the subscripts 2, 4, 10, and 13 indicate the locations of the three base edits reference in FIG. 8, namely, C2T, C→T, C10T, and C13T. The target site was edited using a cytidine deaminase base editor (rAPOBEC BE4) in combination with the guides indicated on the x-axis, which ranged in length from 18 to 23 nucleotides (see Table 1). In FIG. 8 the conversions corresponding to each bar are indicated above each bar using the letter a, b, c, and d, where “a” indicates “C2T,” “b” indicates “C→T,” “c” indicates “C10T,” and “d” indicates “C13T.”

### DETAILED DESCRIPTION

The invention features compositions and methods for editing a 290-KD centrosomal protein (CEP290) gene to treat a congenital eye disorder, such as Leber's Congenital Amaurosis-10. In embodiments, the disclosure provides methods for direct correction of the IVS26 pathogenic mutation in the CEP290 gene (CEP290 c.2991+1655A>G) and/or disruption of a cryptic splice donor site within an intron of the CEP290 gene using a base editor (e.g., a cytidine deaminase base editor, an adenosine deaminase base editor, or a cytidine adenosine deaminase base editor (CABE)).

The invention is based, at least in part, on the discovery, as shown in the Examples provided herein below, that base editors can be used to directly correct the IVS26 pathogenic mutation in the CEP290 gene (CEP290 c.2991+1655A>G) and/or disruption of a cryptic splice donor site within an intron of an CEP290 gene to treat Leber's Congenital Amaurosis. In particular, it was found that precise correction of the IVS26 pathogenic mutation can be achieved using a cytidine deaminase base editor (e.g., a base editor containing a rAPOBEC, ppAPOBEC, RrA3F, AmAPOBEC1, or SsAPOBEC3B cytidine deaminase domain) with limited bystander edits and with editing efficiencies of about 75% in vitro. It was also found that disruption of a cryptic splice site associated with the IVS26 pathogenic mutation could be achieved using a cytidine deaminase base editor (e.g., a base editor containing a rAPOBEC, ppAPOBEC, RrA3F, AmAPOBEC1, or SsAPOBEC3B cytidine deaminase domain) with editing efficiencies of about 35% in vitro with limited or no detectable bystander editing. Base editing efficiencies of as low as 29% have been found to produce a therapeutic effect in mice in the context of editing the Rpe65 gene to treat Leber congenital amaurosis (see, e.g., Suh, et al., “Restoration of visual function in adult mice with an inherited retinal disease via adenine base editing,” Nat Biomed Eng, 5:169-178 (2021), doi: 10.1038/s41551-020-00632-6, the disclosure of which is incorporated herein by reference in its entirety for all purposes).

### Leber's Congenital Amaurosis-10 (LCA10) and CEP290

Leber's Congenital Amaurosis-10 (LCA10) is an autosomal recessive disorder causing severe retinal dystrophy. LCA is inherited in an autosomal recessive fashion and accounts for approximately 5% of all inherited retinal dystrophies. LCA10 is associated with severe visual impairment at birth or early childhood that progresses with age. In some cases, LCA10 is associated with loss of peripheral vision and cones remaining present in the fovea. Since LCA10 is characterized in many cases with a loss of peripheral vision, delayed atrophy or degeneration of cone photoreceptors relative to rod receptors provides a target for a one-time base editing strategy.

Symptoms associated with LCA10 include reduced vision (e.g., extreme farsightedness (hyperopia)), lack of response to visual cues, involuntary roving eye movements (nystagmus), cataracts, corneal abnormality (keratoconus), aversion to light (photophobia), hearing impairment and developmental delays, epilepsy, motor skill impairment, retina deterioration, think or narrow retinal blood vessels, pigmentary changes in the retina, infantile nystagmus, sluggish papillary responses, and/or a paradoxical pupil response. Further, Francechetti's oculo-digital sign is a characteristic of the disease, which involves poking, pressing, and/or tubbing the eyes with a knuckle or finger.

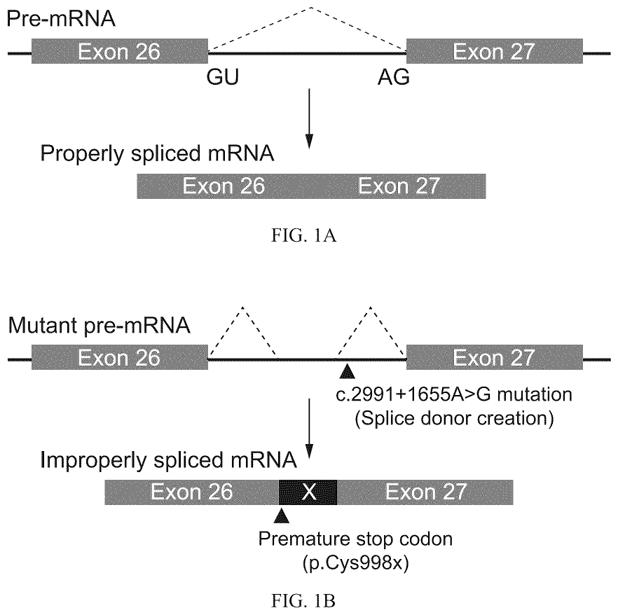

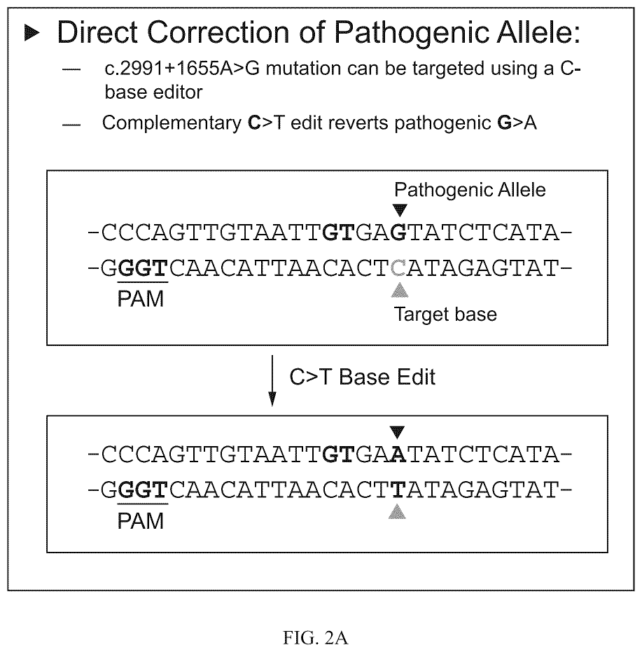

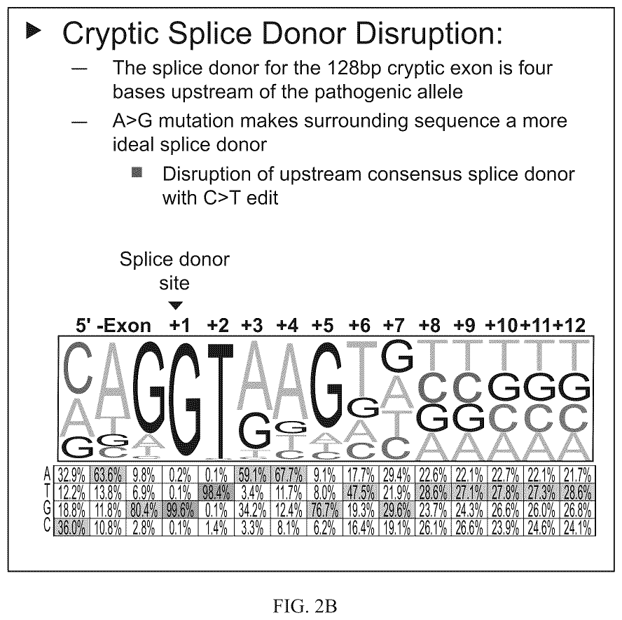

Approximately 2-3 in 100,000 individuals have LCA10. About 20%-30% of LCA10 cases are due to a mutation to CEP290 (FIGS. 1A and 1B). Up to 80% of those cases (˜1,300 patients in the United States) are due to an intronic mutation that creates a cryptic “GT” splice donor site (FIGS. 2A and 2B) and results in a non-functional truncated protein (FIGS. 1A and 1B). In many cases, the pathogenic mutation is c.2991+1655A>G (“the IVS26 mutation”). Therefore, an effective treatment for LCA10 includes either direction correction of the pathogenic allele (FIG. 2A) using a cytidine deaminase base editor or disruption of the cryptic “GT” splice donor site (FIG. 2B) using an adenosine deaminase base editor or a cytidine deaminase base editor. A cytidine deaminase base editor can be used to deaminate the cytidine (C) complementary to the pathogenic guanine (G) to result in a reversion to adenine (i.e., c.2991+1665A). A cytidine deaminase base editor or an adenosine deaminase base editor can be used to alter the cryptic “GT” splice donor site upstream of the c.2991+1665A>G mutation. In some embodiments, a base editor may be used to simultaneously alter the pathogenic target base and one or more bases of the splice donor site (e.g., using a CABE or a cytidine deaminase base editor). One goal of treatment using a base editing strategy includes preventing the retention of a ˜128 bp cryptic exon (IVS26). An advantage of treating LCA10 using a base editing approach is that off-target and unintended edits are reduced relative to alternative strategies (e.g., a CRISPR approach).

The notation “c.2991+1665A” indicates exonic nucleotide 2991, where exonic nucleotide 1 corresponds to the “A” of “ATG” corresponding to the first translated codon of mRNA transcribed from the CEP290 gene (i.e., coding (c.) nucleotide 2991), and +1665A indicates an intronic adenine (A) nucleotide at position 1665 of the intron immediately downstream of exonic nucleotide 2991, where intronic nucleotide number 1 is the first nucleotide 3′ of exonic nucleotide 2991.

290-KD centrosomal protein (CEP290) encodes a widely expressed centrosomal and ciliary protein of 290 kDa that plays an important role in ciliary trafficking and cilium assembly. In the photoreceptors, CEP290 localizes to the connecting cilium, the transitional zone linking the inner and outer segments of rods and cones. Over 100 CEP290 mutations have been identified that lead to a spectrum of phenotypes ranging from isolated early-onset retinal dystrophy and Leber's Congenital Amaurosis-10 to more severe syndromes such as Senior Loken syndrome, Joubert syndrome, or Meckel-Gruber syndrome (MGS). Hypomorphic CEP290 mutations are generally associated with non-syndromic forms of LCA, and account for an estimated 15% of all LCA cases in the Caucasian population. Not intending to be bound by theory, cones are more vulnerable to mutations to CEP290 compared to rods, which may be a consequence of their higher metabolism. Therefore, the progression of vision loss in Leber's Congenital Amaurosis-10 typically begins with the loss of function primarily of cones and proceeds to the loss of function of rods. Not intending to be bound by theory, subsequent to cone degeneration, rod photoreceptor loss occurs in retinal regions characterized by a low density of rods, while the high rod density retinal regions remain intact. Cones are involved in peripheral vision while rods are important for central vision. Therefore, in some embodiments, the methods of the present disclosure result in a preservation of central vision or slowing of the progressive loss thereof in a subject.

### Editing of Target Genes

To produce the gene edits described above (e.g., correction of an IVS26 mutation and/or disruption of a cryptic splice site in CEP290), a subject is administered and/or a cell (e.g., a retinal cell such as a rod cell or a cone cell) is contacted with one or more guide polynucleotides (e.g., one or more of those guide polynucleotides (e.g., guide RNAs) listed in Table 1 or containing one or more of the spacers listed in Table 2, fragments thereof, or 3′ and/or 5′ extensions thereof) and a base editor polypeptide comprising a nucleic acid programmable DNA binding protein (napDNAbp) and a cytidine deaminase or adenosine deaminase, or comprising one or more deaminases with cytidine deaminase and/or adenosine deaminase activity (e.g., a “dual deaminase” which has cytidine and adenosine deaminase activity), or a polynucleotide encoding the same. In embodiments, the base editor and/or endonuclease is introduced to a cell or administered to a subject using a polynucleotide sequence (e.g., mRNA) encoding the base editor and/or endonuclease. In embodiments, the base editor and/or guide RNAs is administered to the subject or contacted with the cell using a suitable vector (e.g., an AAV vector or a lipid nanoparticle). In some cases, the vector targets rods and/or cones. Non-limiting examples of suitable vectors for targeting rods and/or cones include AAV5, PHB.EB, and PHP.B AAV vectors. In some embodiments, the subject is administered and/or the cell is contacted with at least one nucleic acid, wherein the at least one nucleic acid encodes one or more guide RNAs and a nucleobase editor polypeptide comprising a nucleic acid programmable DNA binding protein (napDNAbp) and a cytidine deaminase. In some embodiments, the gRNA comprises nucleotide analogs. In some instances, the gRNA is added directly to a cell. These nucleotide analogs can inhibit degradation of the gRNA from cellular processes. Table 2 provides representative spacer sequences to be used for gRNAs.

Tables 1 and 2 below lists representative guide RNA spacer sequences that can be used in combination with the indicated base editors. Guide RNAs containing the spacer sequences listed in Table 2 can be used to target a base editor (e.g., an adenosine base editor (ABE), a cytidine base editor (CBE), and/or a cytidine adenosine base editor (CABE)) to edit a CEP290 gene. Exemplary spacer sequences suitable for use in gRNA sequences for use in the methods provided herein include fragments of any of the spacers provided in Table 2 as well as any of the spacers provided in Table 2 modified to include an extension or truncation at the 3′ and/or 5′ end(s). In embodiments, a spacer sequence of Table 2 can be modified to include a 1, 2, 3, 4, 5, 6, 7, 8, 9, or 10 nucleotide extension or truncation at the 3′ and/or 5′ end(s).

In various instances, it is advantageous for a spacer sequence to include a 5′ and/or a 3′ “G” nucleotide (e.g., see the lowercase g's in the guide RNAs listed in Table 1 below). In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

In embodiments, a guide polynucleotide provided herein contains a scaffold with about or at least about 85% sequence identity to the following nucleotide sequence:

(SEQ ID NO: 484)
GUUUUAGAGCUAGAAAUAGCAAGUUAAAAUAAGGCUAGUCCGUUAUCAA
CUUGAAAAAGUGGCACCGAGUCGGUGCUUUUUU.

In some cases, a guide polynucleotide of the present disclosure is expressed under the control of a U6 promoter. In some cases, a guide polynucleotide contains the above scaffold and one or more of the spacers listed in Table 2 below, fragments thereof, or 3′ and/or 5′ extensions thereof.

Exemplary guide RNA sequences are provided in the following Tables 1 and 2.

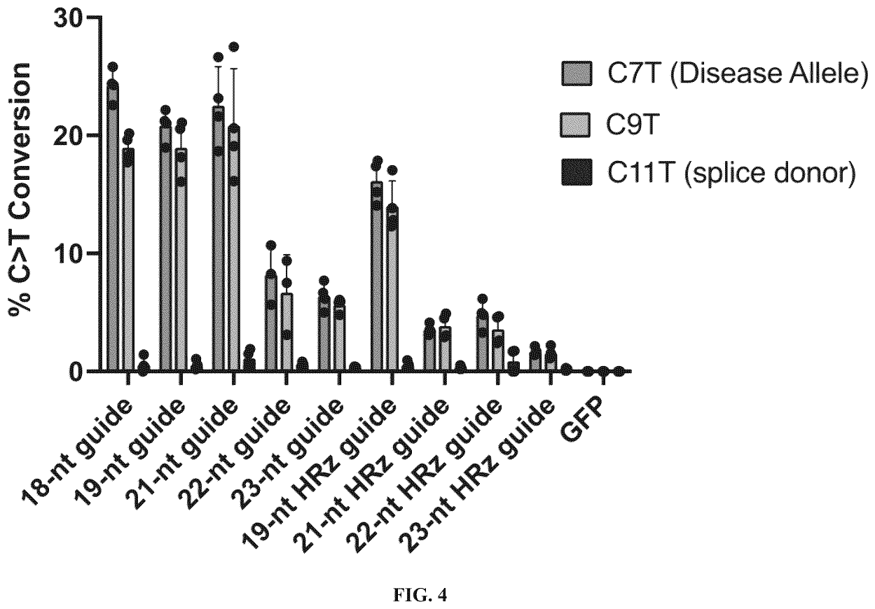

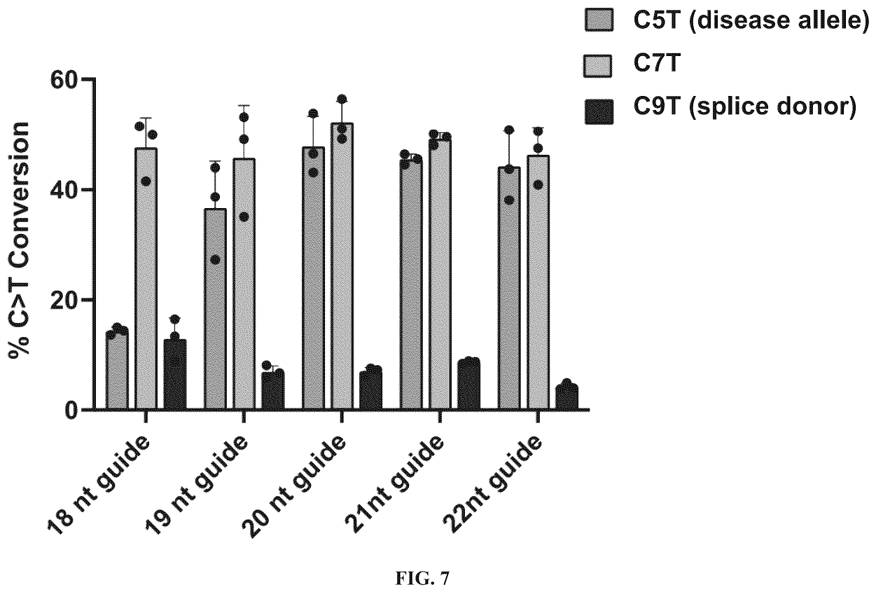

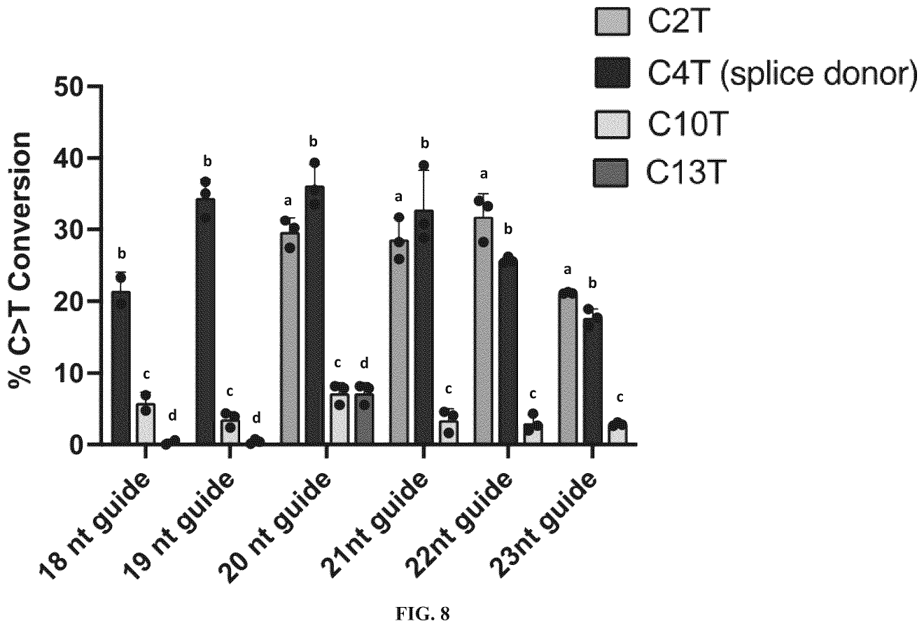

TABLE 1
Exemplary guide polynucleotides

| | gRNA Sequence (scaffold sequence is in bold; | |
|---|---|---|
| | lowercase 5′ “g” indicates a 5′ “G” added to the spacer | |
| Guide name | sequence that is not complementary to a target site; | |
| and | underline indicates a self-cleaving hammerhead | |
| corresponding | ribozyme) (lowercase letters “c” indicate target | SEQ ID |
| FIGS. | nucleotides for base editing) | NO: |
| 18-nt (FIGs. 4- | GAUAcUCACAAUUACAACGUUUUAGAGCUAGAAAUAGCA | 438 |
| 6) | AGUUAAAAUAAGGCUAGUCCGUUAUCAACUUGAAAAAGU | |
| | GGCACCGAGUCGGUGCUUUUUU | |
| 19-nt (FIGs. 4- | gGAUAcUCACAAUUACAACGUUUUAGAGCUAGAAAUAGC | 439 |
| 6) | AAGUUAAAAUAAGGCUAGUCCGUUAUCAACUUGAAAAAG | |
| | UGGCACCGAGUCGGUGCUUUUUU | |
| 20-nt (FIGs. 4- | GAGAUAcUCACAAUUACAACGUUUUAGAGCUAGAAAUAG | 440 |
| 6) | CAAGUUAAAAUAAGGCUAGUCCGUUAUCAACUUGAAAAA | |
| | GUGGCACCGAGUCGGUGCUUUUUU | |
| 21-nt (FIGs. 4- | gGAGAUAcUCACAAUUACAACGUUUUAGAGCUAGAAAUA | 441 |
| 6) | GCAAGUUAAAAUAAGGCUAGUCCGUUAUCAACUUGAAAA | |
| | AGUGGCACCGAGUCGGUGCUUUUUU | |
| 22-nt (FIGs. 4- | gUGAGAUACUCACAAUUACAACGUUUUAGAGCUAGAAAU | 442 |
| 6) | AGCAAGUUAAAAUAAGGCUAGUCCGUUAUCAACUUGAAA | |
| | AAGUGGCACCGAGUCGGUGCUUUUUU | |
| 23-nt (FIGs. 4- | gAUGAGAUAcUCACAAUUACAACGUUUUAGAGCUAGAAA | 443 |
| 6) | UAGCAAGUUAAAAUAAGGCUAGUCCGUUAUCAACUUGAA | |
| | AAAGUGGCACCGAGUCGGUGCUUUUUU | |
| 19-nt HRz | GUAUCUCUGAUGAGUCCGUGAGGACGAAACGAGUAAGCU | 444 |
| (FIGs. 4-6) | CGUCAGAUAcUCACAAUUACAACGUUUUAGAGCUAGAAA | |
| | UAGCAAGUUAAAAUAAGGCUAGUCCGUUAUCAACUUGAA | |
| | AAAGUGGCACCGAGUCGGUGCUUUUUU | |
| 21-nt HRz | gAUCUCACUGAUGAGUCCGUGAGGACGAAACGAGUAAGC | 445 |
| (FIGs. 4-6) | UCGUCUGAGAUAcUCACAAUUACAACGUUUUAGAGCUAG | |
| | AAAUAGCAAGUUAAAAUAAGGCUAGUCCGUUAUCAACUU | |
| | GAAAAAGUGGCACCGAGUCGGUGCUUUUUU | |
| 22-nt HRz | gUAUCUCAUCUGAUGAGUCCGUGAGGACGAAACGAGUAA | 446 |
| (FIGs. 4-6) | GCUCGUCAUGAGAUAcUCACAAUUACAACGUUUUAGAGC | |
| | UAGAAAUAGCAAGUUAAAAUAAGGCUAGUCCGUUAUCAA | |
| | CUUGAAAAAGUGGCACCGAGUCGGUGCUUUUUU | |
| 23-nt HRz | gCUCAUACUGAUGAGUCCGUGAGGACGAAACGAGUAAGC | 447 |
| (FIGs. 4-6) | UCGUCUAUGAGAUAcUCACAAUUACAACGUUUUAGAGCU | |
| | AGAAAUAGCAAGUUAAAAUAAGGCUAGUCCGUUAUCAAC | |
| | UUGAAAAAGUGGCACCGAGUCGGUGCUUUUUU | |
| 18-nt (FIG. 7) | gAcUCACAAUUACAACUGGUUUUAGAGCUAGAAAUAGCA | 448 |
| | AGUUAAAAUAAGGCUAGUCCGUUAUCAACUUGAAAAAGU | |
| | GGCACCGAGUCGGUGCUUUUUU | |
| 19-nt (FIG. 7) | gUAcUCACAAUUACAACUGGUUUUAGAGCUAGAAAUAGC | 449 |
| | AAGUUAAAAUAAGGCUAGUCCGUUAUCAACUUGAAAAAG | |
| | UGGCACCGAGUCGGUGCUUUUUU | |
| 20-nt (FIG. 7) | GAUAcUCACAAUUACAACUGGUUUUAGAGCUAGAAAUAG | 450 |
| | CAAGUUAAAAUAAGGCUAGUCCGUUAUCAACUUGAAAAA | |
| | GUGGCACCGAGUCGGUGCUUUUUU | |
| 21-nt (FIG. 7) | gGAUAcUCACAAUUACAACUGGUUUUAGAGCUAGAAAUA | 451 |
| | GCAAGUUAAAAUAAGGCUAGUCCGUUAUCAACUUGAAAA | |
| | AGUGGCACCGAGUCGGUGCUUUUUU | |
| 22-nt (FIG. 7) | GAGAUAcUCACAAUUACAACUGGUUUUAGAGCUAGAAAU | 452 |
| | AGCAAGUUAAAAUAAGGCUAGUCCGUUAUCAACUUGAAA | |
| | AAGUGGCACCGAGUCGGUGCUUUUUU | |
| 18-nt (FIG. 8) | gCAAUUACAACUGGGGCCGUUUUAGAGCUAGAAAUAGCA | 453 |
| | AGUUAAAAUAAGGCUAGUCCGUUAUCAACUUGAAAAAGU | |
| | GGCACCGAGUCGGUGCUUUUUU | |
| 19-nt (FIG. 8) | gACAAUUACAACUGGGGCCGUUUUAGAGCUAGAAAUAGC | 454 |
| | AAGUUAAAAUAAGGCUAGUCCGUUAUCAACUUGAAAAAG | |
| | UGGCACCGAGUCGGUGCUUUUUU | |
| 20-nt (FIG. 8) | gCACAAUUACAACUGGGGCCGUUUUAGAGCUAGAAAUAG | 455 |
| | CAAGUUAAAAUAAGGCUAGUCCGUUAUCAACUUGAAAAA | |
| | GUGGCACCGAGUCGGUGCUUUUUU | |
| 21-nt (FIG. 8) | gUCACAAUUACAACUGGGGCCGUUUUAGAGCUAGAAAUA | 456 |
| | GCAAGUUAAAAUAAGGCUAGUCCGUUAUCAACUUGAAAA | |
| | AGUGGCACCGAGUCGGUGCUUUUUU | |
| 22-nt (FIG. 8) | gCUCACAAUUACAACUGGGGCCGUUUUAGAGCUAGAAAU | 457 |
| | AGCAAGUUAAAAUAAGGCUAGUCCGUUAUCAACUUGAAA | |
| | AAGUGGCACCGAGUCGGUGCUUUUUU | |
| 23-nt (FIG. 8) | gACUCACAAUUACAACUGGGGCCGUUUUAGAGCUAGAAA | 458 |
| | UAGCAAGUUAAAAUAAGGCUAGUCCGUUAUCAACUUGAA | |
| | AAAGUGGCACCGAGUCGGUGCUUUUUU | |

TABLE 2
Exemplary Spacers

| Guide name | Spacer Sequence (lowercase letters | | |
|---|---|---|---|
| and | indicate target nucleotides for base | | |
| corresponding | editing) or Spacer Sequence Fused to | SEQ | |
| FIGs. | a 5′ HammerHead Ribozyme (HRz) | ID NO | PAM |
| 18-nt (FIGs. | AUACUCACAAUUACAAC | 459 | TGG |
| 4-6) | | | |
| 19-nt (FIGs. | GAUACUCACAAUUACAAC | 460 | TGG |
| 4-6) | | | |
| 20-nt (FIGs. | AGAUACUCACAAUUACAAC | 461 | TGG |
| 4-6) | | | |
| 21-nt (FIGs. | GAGAUACUCACAAUUACAAC | 462 | TGG |
| 4-6) | | | |
| 22-nt (FIGs. | UGAGAUACUCACAAUUACAAC | 463 | TGG |
| 4-6) | | | |
| 23-nt (FIGs. | AUGAGAUACUCACAAUUACAAC | 464 | TGG |
| 4-6) | | | |
| 19-nt HRz | UAUCUCUGAUGAGUCCGUGAGGACGAAACG | 465 | TGG |
| (FIGs. 4-6) | AGUAAGCUCGUCAGAUACUCACAAUUACAA | | |
| | C | | |
| 21-nt HRz | AUCUCACUGAUGAGUCCGUGAGGACGAAAC | 466 | TGG |
| (FIGs. 4-6) | GAGUAAGCUCGUCUGAGAUACUCACAAUUA | | |
| | CAAC | | |
| 22-nt HRz | UAUCUCAUCUGAUGAGUCCGUGAGGACGAA | 467 | TGG |
| (FIGs. 4-6) | ACGAGUAAGCUCGUCAUGAGAUACUCACAA | | |
| | UUACAAC | | |
| 23-nt HRz | CUCAUACUGAUGAGUCCGUGAGGACGAAAC | 468 | TGG |
| (FIGs. 4-6) | GAGUAAGCUCGUCUAUGAGAUACUCACAAU | | |
| | UACAAC | | |
| 18-nt (FIG. 7) | AcUCACAAUUACAACUG | 469 | GGG |
| 19-nt (FIG. 7) | UACUCACAAUUACAACUG | 470 | GGG |
| 20-nt (FIG. 7) | AUACUCACAAUUACAACUG | 471 | GGG |
| 21-nt (FIG. 7) | GAUACUCACAAUUACAACUG | 472 | GGG |
| 22-nt (FIG. 7) | AGAUACUCACAAUUACAACUG | 473 | GGG |
| 18-nt (FIG. 8) | CAAUUACAACUGGGGCC | 474 | AGG |
| 19-nt (FIG. 8) | ACAAUUACAACUGGGGCC | 475 | AGG |
| 20-nt (FIG. 8) | CACAAUUACAACUGGGGCC | 476 | AGG |
| 21-nt (FIG. 8) | UCACAAUUACAACUGGGGCC | 477 | AGG |
| 22-nt (FIG. 8) | CUCACAAUUACAACUGGGGCC | 478 | AGG |
| 23-nt (FIG. 8) | ACUCACAAUUACAACUGGGGCC | 479 | AGG |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects. “Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R.T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity, “Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g. ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)ₙ (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence: ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant
(TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | M | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | T | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | T | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1-m | | | | | | | | | | | | T | | | | | | |
| TadA*8.2-m | | | | | | | | | | | | R | | | | | | |
| TadA*8.3-m | | | | | | | | | | | | | | S | | | | |
| TadA*8.4-m | | | | | | | | | | H | | | | | | | | |
| TadA*8.5-m | | | | | | S | | | | | | | | | | | | |
| TadA*8.6-m | | | | | | | | | | | | | | | | | | R |
| TadA*8.7-m | | | | | | | | | | | | | | R | | | | |
| TadA*8.8-m | | | | | | | | | | H | | R | | R | | | | |
| TadA*8.9-m | | | | | Y | | | | | | | R | | R | | | | |
| TadA*8.10-m | | | | | | | | | | | | R | | R | | | | R |
| TadA*8.11-m | | | | | | | | | | | | T | | R | | | | |
| TadA*8.12-m | | | | | | | | | | | | T | | S | | | | |
| TadA*8.13-m | | | | | Y | | | | | H | | R | | R | | | | |
| TadA*8.14-m | | | | | Y | S | | | | | | | | | | | | |
| TadA*8.15-m | | | | | | S | | | | | | R | | | | | | |
| TadA*8.16-m | | | | | | S | | | | H | | R | | | | | | |
| TadA*8.17-m | | | | | | S | | | | | | | | R | | | | |
| TadA*8.18-m | | | | | | S | | | | H | | | | R | | | | |
| TadA*8.19-m | | | | | | S | | | | H | | R | | R | | | | |
| TadA*8.20-m | | | | | Y | S | | | | H | | R | | R | | | | |
| TadA*8.21-m | | | | | | | | | | | | R | | S | | | | |
| TadA*8.22-m | | | | | | S | | | | | | | | S | | | | |
| TadA*8.23-m | | | | | | S | | | | H | | | | | | | | |
| TadA*8.24-m | | | | | | S | | | | H | | T | | | | | | |
| TadA*8.1-d | | | | | | | | | | | | T | | | | | | |
| TadA*8.2-d | | | | | | | | | | | | R | | | | | | |
| TadA*8.3-d | | | | | | | | | | | | | | S | | | | |
| TadA*8.4-d | | | | | | | | | | H | | | | | | | | |
| TadA*8.5-d | | | | | | S | | | | | | | | | | | | |
| TadA*8.6-d | | | | | | | | | | | | | | | | | | R |
| TadA*8.7-d | | | | | | | | | | | | | | R | | | | |
| TadA*8.8-d | | | | | | | | | | H | | R | | R | | | | |
| TadA*8.9-d | | | | | Y | | | | | | | R | | R | | | | |
| TadA*8.10-d | | | | | | | | | | | | R | | R | | | | R |
| TadA*8.11-d | | | | | | | | | | | | T | | R | | | | |
| TadA*8.12-d | | | | | | | | | | | | T | | S | | | | |
| TadA*8.13-d | | | | | Y | | | | | H | | R | | R | | | | |
| TadA*8.14-d | | | | | Y | S | | | | | | | | | | | | |
| TadA*8.15-d | | | | | | S | | | | | | R | | | | | | |
| TadA*8.16-d | | | | | | S | | | | H | | R | | | | | | |
| TadA*8.17-d | | | | | | S | | | | | | | | R | | | | |
| TadA*8.18-d | | | | | | S | | | | H | | | | R | | | | |
| TadA*8.19-d | | | | | | S | | | | H | | R | | R | | | | |
| TadA*8.20-d | | | | | Y | S | | | | H | | R | | R | | | | |
| TadA*8.21-d | | | | | | | | | | | | R | | S | | | | |
| TadA*8.22-d | | | | | | S | | | | | | | | S | | | | |
| TadA*8.23-d | | | | | | S | | | | H | | | | | | | | |
| TadA*8.24-d | | | | | | S | | | | H | | T | | | | | | |

TABLE 5C
Adenosine Deaminase Variants. Alterations are referenced
to TadA*7.10. Additional details of TadA*9 adenosine
deaminases are described in International PCT Application
No. PCT/US2020/049975, which is incorporated herein
by reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises one or more of M1I, S2A, S2E, W4D, W4E, V4M, F6S, H8E, H8Y, E9Y, M12S, R13H, R13I, R13Y, T17L, T17S, L18A, L18E, A19N, R21N, K20K, K20R, R21A, G22P, W23), R23H, W23G, W23Q, W23L, W23R, D24E, D24G, E25F, E25M, E25D, E25A, E25G, E25R, E25V, E25S, E25Y, R26D, R26E, R26G, R26N, R26Q, R26C, R26L, R26K, R26W, E27V, E27D, P29V, V30G, L34S, L34V, L36H, H36L, H36N, N37N, N37T, N37S, N38G, N38R, W45A, W45L, W45N, N46N, R46W, R46F, R46Q, R46M, R47A, R47Q, R47F, R47K, R47P, R47W, R47M, P48T, P48L, P48A, P48I, P48S, I49G, I49H, I49V, I49F, I49H, G50L, R51H, R51L, R51N, L51W, R51Y, H52D, H52Y, D53P, P54C, P54T, A55H, T55A, A56E, A56S, E59A, E59G, E591, E59Q, E59W, M61A, M61I, M61L, M61V, L63S, L63V, Q65V, G66C, G67D, G67L, G67V, L68Q, M70H, M70Q, L84F, M70V, M70L, E70A, M70V, Q71M, Q71N, Q71L, Q71R, N72A, N72K, N72S, N72D, N72Y, Y73G, Y73I, Y73K, Y73R, Y73S, R74A, R74Q, R74G, R74K, R74L, R74N, I76D, I76F, I76I, I76N, I76T, I76Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, I95S, I95G, I95L, I95H, I95V, H96A, H96L, H96R, H96S, S97C, S97G, S97I, S97M, S97R, S97S, R98K, R98I, R98N, R98Q, G100R, G100V, R101V, R101R, V102A, V102F, V102I, V102V, D103A, F104G, D104N, F104V, F104I, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K110R, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P1241, P124L, P124W, G125H, G125I, G125A, G125M, G125K, M126D, M126H, M126K, M126I, M126N, M126O, M126S, M126Y, N127H, N127S, N127D, N127K, N127R, H128R, R129H, R129Q, R129V, R129I, R129E, R129V, I132I, I132F, T133V, T133E, T133G, T133K, E134A, E134E, E134G, E134I, G135G, G135V, I136G, I136L, I136T, I137A, I137D, I137E, L137M, I137S, A138D, A138E, A138G, S138A, A138N, A138S, A138T, A138V, A138Y, D139E, D1391, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E155I, E155K, E155P, E155V, E155D, I156A, I156F, I156D, I156K, I156N, I156R, I156Y, E157A, E157F, E157I, E157P, E157T, E157V, N157K, K157N, K157R, A158Q, A158K, A158V, Q159F, Q159K, Q159L, Q159N, K160A, K160S, K160E, K160K, K160N, K161I, K161A, K161N, K161Q, K161S, K161T, A162D, A162Q, R162H, R162P, A162S, Q163G, Q163H, Q163N, Q163R, S1641, S164R, S164Y, S165A, S165D, S165I, S165T, S165Y, T166D, T166K, T166I, T166N, T166P, T166R, D167S and/or D167N mutation in a TadA reference sequence (e.g., TadA*7.10,ecTadA, or TadA8e), and any alternative mutation at the corresponding position, or any substitution from R26, W23, E27, H36, R47, P48, R51, H52, R74, I76, V82, V88, M94, I95, H96, A106, D108, A109, K110, T111, A114, D119, H122, H123, M126, N127, A142, S146, D147, F149, R152, Q154, E155, I156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, I156F, K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the invention comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the invention comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N.M., et al., “Programmable base editing of A⋅T to G⋅C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the invention comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the invention comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (TADC)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the invention is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+I49N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 301, 408, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to
TadA*8.20. “S” indicates “Surface,” and “NAS” indicates “Near Active Site.”

| location in structure | N/A | S h1 | S h1 | S h1 | NAS | NAS | NAS | NAS | | S | |
|---|---|---|---|---|---|---|---|---|---|---|---|
| Amino Acid | 2 | 8 | 13 | 17 | 27 | 47 | 48 | 49 | 67 | 76 | 77 |
| No. (*START | | | | | | | | | | | |
| Met is AA#1) | | | | | | | | | | | |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | T | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

Adenosine Deaminase Variants. Mutations are indicated with
reference to TadA*8.20. “I” indicates “Internal,” “S” indicates “Surface,” and “NAS”
indicates “Near Active Site.”

| location in structure | I | NAS | | NAS | S | | S | S | S | | S | |
|---|---|---|---|---|---|---|---|---|---|---|---|---|
| Amino Acid | 82 | 84 | 96 | 107 | 112 | 115 | 118 | 119 | 127 | 142 | 162 | 165 |
| No. (*START | | | | | | | | | | | | |
| Met is AA#1) | | | | | | | | | | | | |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are indicated with
reference to TadA*8.20.

| Position No. | 27 | 29 | 30 | 49 | 82 | 84 | 107 | 112 | 115 | 142 |
|---|---|---|---|---|---|---|---|---|---|---|
| TadA*8.20 | E | P | V | I | S | F | R | G | G | A |
| Alterations | | | | | | | | | | |
| Evaluated | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | | H | M | E |

TABLE 6C
Adenosine deaminase variants. Mutations are indicated with reference to
variant 1.2 (Table 6A) .

| | | Residue identity (START Met is amino | | | | | | | | |
|---|---|---|---|---|---|---|---|---|---|---|
| | | acid #1) | | | | | | | | |
| Variant Name | Alternative Variant Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |
| | | Residue identity (START Met is | | | | | | | | |
| | | amino acid #1) | | | | | | | | |
| Variant Name | Alternative Variant Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to
TadA*8.20.

| AA Positions | 6 | 27 | 49 | 76 | 77 | 82 | 107 | 112 | 114 | 115 | 119 | 122 | 127 | 142 | 143 |
|---|---|---|---|---|---|---|---|---|---|---|---|---|---|---|---|
| TadA*8.20 | F | E | I | Y | D | S | R | G | A | G | D | H | N | A | A |
| S1.154 | F | H | K | Y | D | T | C | H | | M | | | | E | |
| Alterations from Table 6C | Y | | | W | G | | | | C | | N | G | P | | E |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to
TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19 m/8.20 m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 511. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation.

In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows: PKKKRKVEGADKRTADGSE FES PKKKRKV (SEQ ID NO: 328)

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Coin RNA binding protein domain (e.g. SfMu Coin coat protein domain, and SfMu Coin binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and VoB, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + |
| | | Q154S |
| ABE-680m | MSP680 | monomer_TadA*7.10 + I76Y + V82G + |
| | | Y147T + Q154S |
| ABE-823m | MSP823 | monomer_TadA*7.10 + L36H + V82G + |
| | | Y147T + Q154S + N157K |
| ABE-824m | MSP824 | monomer_TadA*7.10 + V82G + Y147D + |
| | | F149Y + Q154S + D167N |
| ABE-825m | MSP825 | monomer_TadA*7.10 + L36H + V82G + |
| | | Y147D + F149Y + Q154S + N157K + |
| | | D167N |
| ABE-827m | MSP827 | monomer_TadA*7.10 + L36H + I76Y + |
| | | V82G + Y147T + Q154S + N157K |
| ABE-828m | MSP828 | monomer_TadA*7.10 + I76Y + V82G + |
| | | Y147D + F149Y + Q154S + D167N |
| ABE-829m | MSP829 | monomer_TadA*7.10 + L36H + I76Y + |
| | | V82G + Y147D + F149Y + Q154S + |
| | | N157K + D167N |
| ABE-605d | MSP605 | heterodimer_(WT) + (TadA*7.10 + V82G + |
| | | Y147T + Q154S) |
| ABE-680d | MSP680 | heterodimer_(WT) + (TadA*7.10 + I76Y + |
| | | V82G + Y147T + Q154S) |
| ABE-823d | MSP823 | heterodimer_(WT) + (TadA*7.10 + L36H + |
| | | V82G + Y147T + Q154S + N157K) |
| ABE-824d | MSP824 | heterodimer_(WT) + (TadA*7.10 + V82G + |
| | | Y147D + F149Y + Q154S + D167N) |
| ABE-825d | MSP825 | heterodimer_(WT) + (TadA*7.10 + L36H + |
| | | V82G + Y147D + F149Y + Q154S + |
| | | N157K + D167N) |
| ABE-827d | MSP827 | heterodimer_(WT) + (TadA*7.10 + L36H + |
| | | I76Y + V82G + Y147T + Q154S + N157K) |
| ABE-828d | MSP828 | heterodimer_(WT) + (TadA*7.10 + I76Y + |
| | | V82G + Y147D + F149Y + Q154S + |
| | | D167N) |
| ABE-829d | MSP829 | heterodimer_(WT) + (TadA*7.10 + L36H + |
| | | I76Y + V82G + Y147D + F149Y + |
| | | Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)ₙ motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence:

(SEQ ID NO: 361)
SGGSSGGSSGSETPGTSESATPESSGGSSGGSSGGSSGGSSGSETPGTS
ESATPESSGGSSGGS.

In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.
Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the invention is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the invention advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The invention provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the base editor systems provided herein result in less than 70%, less than 65%, less than 60%, less than 55%, 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% bystander editing of one or more nucleotides (e.g., mutation of an off-target nucleotide). In embodiments, the bystander nucleotide(s) are selected from one or more of C9 and/or C11 of the following sequence ATAC₇TC₉AC₁₁AATTACAACTGG (SEQ ID NO: 481) and/or C2, C10, and/or C13 of the following sequence CTC₂AC₄AATTAC₁₀AAC₁₃TGGGGCC (SEQ ID NO: 483) and/or C5 and/or C7 of the following sequence AC₅TC₇AC₉AATTACAACTGGGG (SEQ ID NO: 482).

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N.M., et al., “Programmable base editing of A⋅T to G⋅C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the invention comprising a deaminase may be expressed in virtually any host cell of interest, including but not limited to animal cells using routine methods known to the skilled artisan. For example, a DNA encoding an adenosine deaminase of the invention can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa.or.jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present invention, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like are preferable.

Any suitable promoter can be used to drive expression of a base editor system or component thereof and, where appropriate, the guide nucleic acid. For ubiquitous expression, promoters include CMV, CBA, CBH, CAG, CBh, PGK, SV40, Ferritin heavy or light chains. In some embodiments, the promoter facilitates or preferentially facilitates expression (e.g., cell-type specific expression) in retinal cells, such as rod and/or cone cells. For cone cell expression, suitable promoters include PR1.7, hG1.7, and hGRK 198 bp, exemplary nucleotide sequences for which include those with at least 85% sequence identity to one of the following representative nucleotide sequences and capable of promoting the expression of a gene in a cell:

Representative PR1.7 promoter sequence (GenBank Accession No. KT886395.1):
source:
(SEQ ID NO: 485)
GGAGGCTGAGGGGTGGGGAAAGGGCATGGGTGTTTCATGAGGACAGAGCTTCCGTTTCATGC
AATGAAAAGAGTTTGGAGACGGATGGTGGTGACTGGACTATACACTTACACACGGTAGCGAT
GGTACACTTTGTATTATGTATATTTTACCACGATCTTTTTAAAGTGTCAAAGGCAAATGGCC
AAATGGTTCCTTGTCCTATAGCTGTAGCAGCCATCGGCTGTTAGTGACAAAGCCCCTGAGTC
AAGATGACAGCAGCCCCCATAACTCCTAATCGGCTCTCCCGCGTGGAGTCATTTAGGAGTAG
TCGCATTAGAGACAAGTCCAACATCTAATCTTCCACCCTGGCCAGGGCCCCAGCTGGCAGCG
AGGGTGGGAGACTCCGGGCAGAGCAGAGGGCGCTGACATTGGGGCCCGGCCTGGCTTGGGTC
CCTCTGGCCTTTCCCCAGGGGCCCTCTTTCCTTGGGGCTTTCTTGGGCCGCCACTGCTCCCG
CTCCTCTCCCCCCATCCCACCCCCTCACCCCCTCGTTCTTCATATCCTTCTCTAGTGCTCCC
TCCACTTTCATCCACCCTTCTGCAAGAGTGTGGGACCACAAATGAGTTTTCACCTGGCCTGG
GGACACACGTGCCCCCACAGGTGCTGAGTGACTTTCTAGGACAGTAATCTGCTTTAGGCTAA
AATGGGACTTGATCTTCTGTTAGCCCTAATCATCAATTAGCAGAGCCGGTGAAGGTGCAGAA
CCTACCGCCTTTCCAGGCCTCCTCCCACCTCTGCCACCTCCACTCTCCTTCCTGGGATGTGG
GGGCTGGCACACGTGTGGCCCAGGGCATTGGTGGGATTGCACTGAGCTGGGTCATTAGCGTA
ATCCTGGACAAGGGCAGACAGGGCGAGCGGAGGGCCAGCTCCGGGGCTCAGGCAAGGCTGGG
GGCTTCCCCCAGACACCCCACTCCTCCTCTGCTGGACCCCCACTTCATAGGGCACTTCGTGT
TCTCAAAGGGCTTCCAAATAGCATGGTGGCCTTGGATGCCCAGGGAAGCCTCAGAGTTGCTT
ATCTCCCTCTAGACAGAAGGGGAATCTCGGTCAAGAGGGAGAGGTCGCCCTGTTCAAGGCCA
CCCAGCCAGCTCATGGCGGTAATGGGACAAGGCTGGCCAGCCATCCCACCCTCAGAAGGGAC
CCGGTGGGGCAGGTGATCTCAGAGGAGGCTCACTTCTGGGTCTCACATTCTTGGATCCGGTT
CCAGGCCTCGGCCCTAAATAGTCTCCCTGGGCTTTCAAGAGAACCACATGAGAAAGGAGGAT
TCGGGCTCTGAGCAGTTTCACCACCCACCCCCCAGTCTGCAAATCCTGACCCGTGGGTCCAC
CTGCCCCAAAGGCGGACGCAGGACAGTAGAAGGGAACAGAGAACACATAAACACAGAGAGGG
CCACAGCGGCTCCCACAGTCACCGCCACCTTCCTGGCGGGGATGGGTGGGGCGTCTGAGTTT
GGTTCCCAGCAAATCCCTCTGAGCCGCCCTTGCGGGCTCGCCTCAGGAGCAGGGGAGCAAGA
GGTGGGAGGAGGAGGTCTAAGTCCCAGGCCCAATTAAGAGATCAGGTAGTGTAGGGTTTGGG
AGCTTTTAAGGTGAAGAGGCCCGGGCTGATCCCACAGGCCAGTATAAAGCGCCGTGACCCTC
AGGTGATGCGCCAGGGCCGGCTGCCGTCGGGGACAGGGCTTTCCATAGCCATG.
Representative hG1.7 promoter sequence (see U.S. Patent Application
Publicatin No. 20200392536, the disclosure of which is incorporated herein
by reference in its entirety for all purposes):
(SEQ ID NO: 486)
TAGGAATAGAAGGGTGGGTGCAGGAGGCTGAGGGGTGGGGAAAGGGCATGGGTGTTTCATGA
GGACAGAGCTTCCGTTTCATGCAATGAAAAGAGTTTGGAGACGGATGGTGGTGACTGGACTA
TACACTTACACACGGTAGCGATGGTACACTTTGTATTATGTATATTTTACCACGATCTTTTT
AAAGTGTCAAAGGCAAATGGCCAAATGGTTCCTTGTCCTATAGCTGTAGCAGCCATCGGCTG
TTAGTGACAAAGCCCCTGAGTCAAGATGACAGCAGCCCCCATAACTCCTAATCGGCTCTCCC
GCGTGGAGTCATTTAGGAGTAGTCGCATTAGAGACAAGTCCAACATCTAATCTTCCACCCTG
GCCAGGGCCCCAGCTGGCAGCGAGGGTGGGAGACTCCGGGCAGAGCAGAGGGCGCTGACATT
GGGGCCCGGCCTGGCTTGGGTCCCTCTGGCCTTTCCCCAGGGGCCCTCTTTCCTTGGGGCTT
TCTTGGGCCGCCACTGCTCCCGCTCCTCTCCCCCCATCCCACCCCCTCACCCCCTCGTTCTT
CATATCCTTCTCTAGTGCTCCCTCCACTTTCATCCACCCTTCTGCAAGAGTGTGGGACCACA
AATGAGTTTTCACCTGGCCTGGGGACACACGTGCCCCCACAGGTGCTGAGTGACTTTCTAGG
ACAGTAATCTGCTTTAGGCTAAAATGGGACTTGATCTTCTGTTAGCCCTAATCATCAATTAG
CAGAGCCGGTGAAGGTGCAGAACCTACCGCCTTTCCAGGCCTCCTCCCACCTCTGCCACCTC
CACTCTCCTTCCTGGGATGTGGGGGCTGGCACACGTGTGGCCCAGGGCATTGGTGGGATTGC
ACTGAGCTGGGTCATTAGCGTAATCCTGGACAAGGGCAGACAGGGCGAGCGGAGGGCCAGCT
CCGGGGCTCAGGCAAGGCTGGGGGCTTCCCCCAGACACCCCACTCCTCCTCTGCTGGACCCC
CACTTCATAGGGCACTTCGTGTTCTCAAAGGGCTTCCAAATAGCATGGTGGCCTTGGATGCC
CAGGGAAGCCTCAGAGTTGCTTATCTCCCTCTAGACAGAAGGGGAATCTCGGTCAAGAGGGA
GAGGTCGCCCTGTTCAAGGCCACCCAGCCAGCTCATGGCGGTAATGGGACAAGGCTGGCCAG
CCATCCCACCCTCAGAAGGGACCCGGTGGGGCAGGTGATCTCAGAGGAGGCTCACTTCTGGG
TCTCACATTCTTGACAGGTATTTGCCACTAAGCCCAGCTAATTGTTTTTTATTTAGTAGAAA
CGGGGTTTCACCATGTTAGTCAGGCTGGTCGGGAACTCCTGACCTCAGGAGATCTACCCGCC
TTGGCCTCCCAAAGTGCTGGGATTACAGGCGTGTGCCACTGTGCCCAGCCACTTTTTTTTAG
ACAGAGTCTTGGTCTGTTGCCCAGGCTAGAGTTCAGTGGCGCCATCTCAGCTCACTGCAACC
TCCGCCTCCCAGATTCAAGCGATTCTCCTGCCTCGACCTCCCAGTAGCTGGGATTACAGGTT
TCCAGCAAATCCCTCTGAGCCGCCCCCGGGGGCTCGCCTCAGGAGCAAGGAAGCAAGGGGTG
GGAGGAGGAGGTCTAAGTCCCAGGCCCAATTAAGAGATCAGATGGTGTAGGATTTGGGAGCT
TTTAAGGTGAAGAGGCCCGGGCTGATCCCACTGGCCGGTATAAAGCACCGTGACCCTCAGGT
GACGCACCATCTAGAGCTGCCGTCGGGGACAGGGCTTTCCATAGCC.
Representative hGRK 198 bp promoer sequence:
(SEQ ID NO: 487)
GGGCCCCAGAAGCCTGGTggttgtttgtccttctcaggggaaaagtgaggcggccccttgga
ggaaggggccgggcagaatgatctaatcggattccaagcagctcaggggattgtctttttct
agcaccttcttgccactcctaagcgtcctccgtgaccccggctgggatttagcctggtgctg
tgtcagccccgg.

For brain or other CNS cell expression, suitable promoters include: SynapsinI for all neurons, CaMKIIalpha for excitatory neurons, GAD67 or GAD65 or VGAT for GABAergic neurons. For liver cell expression, suitable promoters include the Albumin promoter. For lung cell expression, suitable promoters include SP-B. For endothelial cells, suitable promoters include ICAM. For hematopoietic cell expression suitable promoters include IFNbeta or CD45. For osteoblast expression suitable promoters can include OG-2.

Expression vectors for use in the present invention, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the invention can be intracellularly expressed by introducing into the cell an expression vector comprising a nucleic acid sequence encoding the fusion protein or complex. In embodiments, a fusion protein or complex of the invention is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (QssDNA) donors.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N(e.g., split intein-N) and Cfa-C(e.g., split intein-C) intein pair, which has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377, 389-402 and 489-510. Inteins suitable for use in embodiments of the present disclosure and methods for use thereof are described in U.S. Pat. No. 10,526,401, International Patent Application Publication No. WO 2013/045632 or WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### Pharmaceutical Compositions

In some aspects, the present invention provides a pharmaceutical composition comprising any of the polynucleotides, vectors, base editors, base editor systems, guide polynucleotides (e.g., the gRNAs listed in Table 1 or guide polynucleotides containing any of the spacers listed in Table 2, or fragments or extensions thereof), fusion proteins, complexes, or the fusion protein-guide polynucleotide complexes described herein.

The pharmaceutical compositions of the present invention can be prepared in accordance with known techniques. See, e.g., Remington, The Science and Practice of Pharmacy (21st ed. 2005). In general, a base editor, base editory system, guide RNA(s), or polynucleotides encoding the same, is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., an eye, such as the retina of an eye). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present invention provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising a base editor or a polynucleotide encoding the base editor (e.g., a CBE, ABE, or CABE) and a guide polynucleotide (e.g., a guide RNA listed in Table 1 or a guide RNA containing a spacer listed in Table 2) of the present disclosure. In other embodiments, the methods of the invention comprise expressing or introducing into a cell a base editor polypeptide and one or more guide RNAs capable of targeting a nucleic acid molecule encoding at least one polypeptide.

The methods herein include administering to a subject (including a subject identified as being in need of such treatment, or a subject suspected of being at risk of disease and in need of such treatment) an effective amount of a composition described herein. In various embodiments, a composition described herein is administered to a subject by subretinal or subfovial injection. In some instances, subretinal injection involves the formation of a bleb in the fovea. Identifying a subject in need of such treatment can be in the judgment of a subject or a health care professional and can be subjective (e.g., opinion) or objective (e.g., measurable by a test or diagnostic method).

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

Provided herein are methods of treating a disease or disorder with a composition or system, e.g., a base editor system or a base editor protein, described herein. Optionally, the base editor system described herein can be combined with one or more other treatments. In one aspect, provided herein is a method of treating Leber's Congenital Amaurosis-10 (LCA10) in a subject in need thereof by administering a base editor described herein, or a polynucleotide(s) encoding the same.

The response in individual subjects can be characterized as a complete response, a partial response, or stable disease. In some embodiments, the response is a partial response (PR). In some embodiments, the response is a complete response (CR). In some embodiments, the response results in preserved or improved vision of the subject (e.g., stable disease).

In some embodiments, the treatment results in improved or preserved vision over time in the human subject as compared to the expected survival time of the human subject if the human subject was not treated with the compound, e.g. the base editor system. In some embodiments preserved vision involves slowed progression of the disease compared to a subject not treated with the compositions or according to the methods provided herein.

In some embodiments, the human subject to be treated with the described methods is a child (e.g., 0-18 years of age). In other embodiments, the human subject to be treated with the described methods is an adult (e.g., 18+ years of age). In some embodiments, the subject is a neonate. In some embodiments, the subject is about or at least about 0 days, 1 day, 2 days, 3 days, 4 days, 5 days, 6 days, 7 days, 1 month, 2 months, 3 months, 4 months, 5 months, 6 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 30 years, or 40 years old. In some embodiments, the subject is no more than about 1 day, 2 days, 3 days, 4 days, 5 days, 6 days, 7 days, 1 month, 2 months, 3 months, 4 months, 5 months, 6 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 30 years, or 40 years old.

### Kits

The invention provides kits for the treatment of Leber's Congenital Amaurosis-10 (LCA10) in a subject. In some embodiments, the kit includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase.

The kits may further comprise written instructions for using a base editor or base editor system as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Screening of Guides for Use in Direct Correction of the Leber's Congenital Amaurosis-10 (LCA10) IVS26 Mutation (CEP290 c.2991+1655A>G)

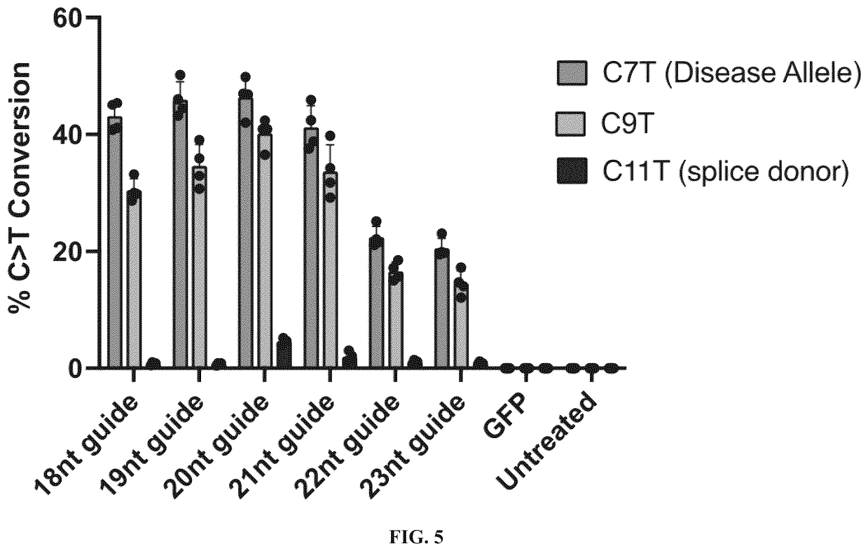

Experiments were undertaken to evaluate the feasibility of using base editing to directly correct the pathogenic IVS26 mutation (CEP290 c.2991+1655A>G) mutation by deaminating the cytidine (C) complementary to the pathogenic guanine (G). Guide RNAs were prepared for targeting a rat APOBEC (rAPOBEC) BE4 cytidine base editor to deaminate the target cytidine (i.e., the cytidine (C) labeled with the subscript “7” in the following sequence: ATAC₇TC₉AC₁₁AATTACAACTGG (SEQ ID NO: 481)). Guide RNAs were evaluated that had lengths of 18, 19, 21, 22, or 23 nucleotides, and guides were also evaluated that contained spacers of the same lengths and also a self-cleaving hammerhead ribozyme (HRz) (FIG. 4 and Table 1). In a first base editing experiment, base editing was evaluated in IVS26 lenti-integrated HEK293T cells (see below Methods). The IVS26 lenti-integrated HEK293T cells were contacted with a plasmid encoding a rat APOBEC (rAPOBEC) BE4 editor expressed under the control of a CMV promoter and with a plasmid encoding a guide RNA expressed under the control of a U6 promoter. The guide RNAs containing spacers with lengths of 18, 19, and 21 nucleotides performed the best, achieving percent C>T conversions of ˜25% (FIG. 4). The base editing experiment was repeated in a second base editing experiment with the inclusion of a guide RNA containing a 20 nucleotide spacer and omitting the guides containing the hammerhead ribozymes (HR_{ZS}) (FIG. 5). This second base editing experiment resulted in overall editing efficiencies of around 45% for the guide RNAs containing 18, 19, 20 and 21 nucleotide spacers.

### Example 2: Screening of Cytidine Deaminase Base Editors for Use in Direct Correction of the Leber's Congenital Amaurosis-10 (LCA10) IVS26 Mutation (CEP290 c.2991+1655A>G)

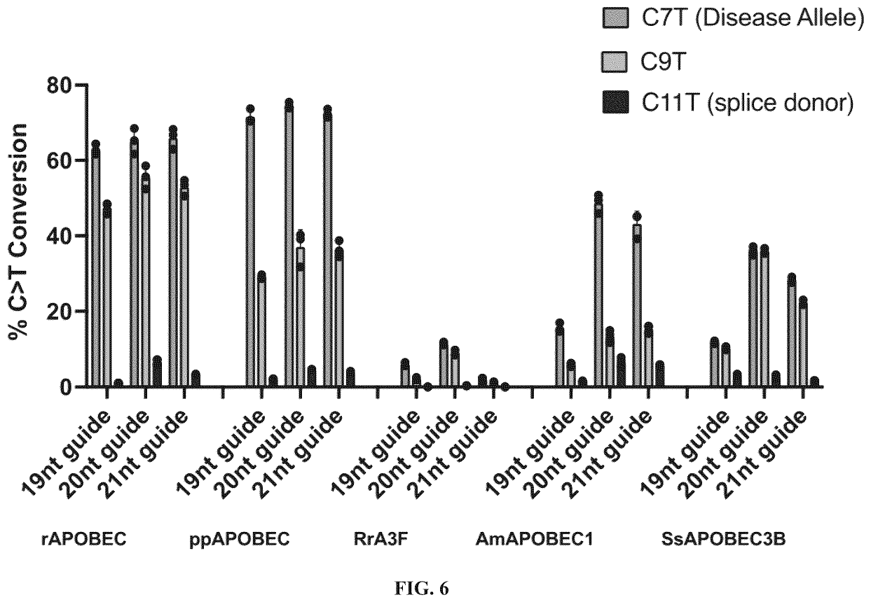

Experiments were undertaken to evaluate five different BE4 cytidine deaminase base editors each containing a different cytidine deaminase domain, namely rAPOBEC, ppAPOBEC, RrA3F, AmAPOBEC1, or SsAPOBEC3B, where “rAPOBEC” indicates a base editor containing “rat APOBEC,” “ppAPOBEC” indicates a base editor containing “Pongo pygmaeus (Orangutan) APOBEC,” RrA3F indicates a base editor containing “Rhinopithecus roxellana (golden snub-nosed monkey) APOBEC3F (A3F),” “AmAPOBEC1” indicates a base editor containing “Alligator mississippiensis (American alligator) APOBEC1,” and SsAPOBEC3B indicates a base editor containing “Sus scrofa (pig) APOBEC3B” (see Yu, Y., et al. (2020). Cytosine base editors with minimized unguided DNA and RNA off-target events and high on-target activity. Nat Commun, 11:2052, the disclosure of which is incorporated herein by reference in its entirety for all purposes). Off-target base bystander base editing was compared between the five BE4 cytidine deaminase base editors. Guide RNAs were prepared for targeting the BE4 cytidine deaminase base editors to deaminate a target cytidine (i.e., the cytidine (C) labeled with the subscript “7” in the following sequence: ATAC₇TC₉AC₁₁AATTACAACTGG (SEQ ID NO: 481), where locations of the off-target C9T and C11T base edits are indicated by the subscripts 9 and 11, respectively). The guides contained spacers with lengths of 19, 20, or 21 nucleotides (see FIG. 6 and Table 1). An optimal base editor would minimize C9T bystander edits while maintaining or increasing target C7T base edits.

The BE4 cytidine deaminases were each introduced to IVS26 lenti-integrated HEK293T cells as split base editors containing a Cfa(GEP) split intein, where each editor was split between the amino acid residues corresponding Glu573 and Cys574 of Cas9. The split editors were encoded by two separate plasmids and the IVS26 lenti-integrated HEK293T cells were co-transfected with two plasmids collectively encoding each split base editor. Each portion/split of each split base editor was expressed from a CMV promoter. The guide RNA used with each split base editor was encoded under the control of a U6 promoter on the vector that also encoded the C-terminal split of each base editor.

The ppAPOBEC, RrA3F, AmAPOBEC1, and SsAPOBEC3B BE4 base editors had reduced bystander C9T editing relative to the rAPOBEC BE4 base editor (FIG. 6). In particular, the ppAPOBEC base editor showed reduced levels of the bystander C9T edit (55%>35%) and improved on-target C7T editing (66%>75%).

### Example 3: Screening of Guides for Use in Disruption of a Cryptic Splice Donor Site Upstream of the IVS26 Mutation (CEP290 c.2991+1655A>G) to Treat Leber's Congenital Amaurosis-10 (LCA10)

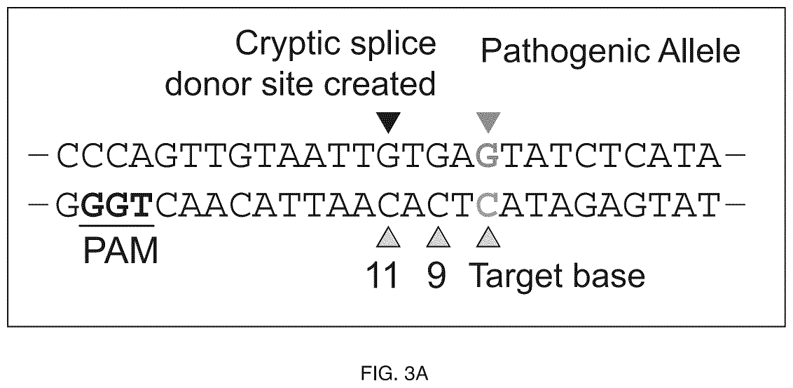

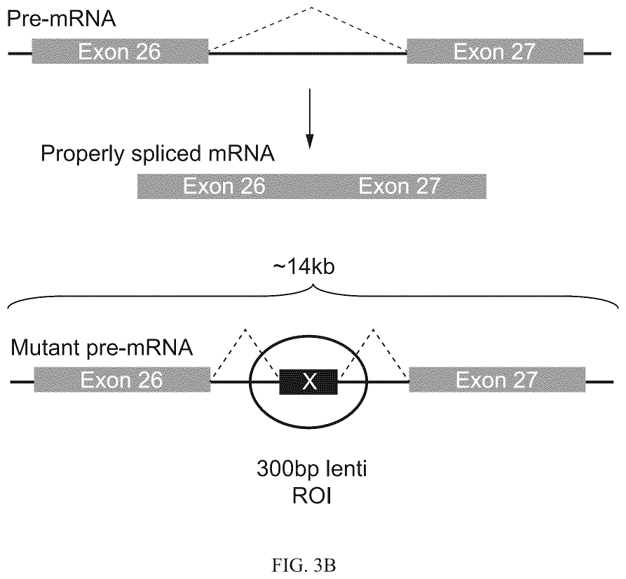

Experiments were undertaken to evaluate the feasibility of using base editing to disrupt a cryptic splice donor site upstream of the IVS26 Mutation (CEP290 c.2991+1655A>G). First, two NGG PAM sequences were identified near the cryptic splice donor site (FIGS. 2B and 3A) upstream of the IVS26 that would allow for targeting an rAPOBEC BE4 cytidine deaminase base editor to deaminate C9 in the following sequence AC₅TC₇AC₉AATTACAACTGGGG (Site 1; SEQ ID NO: 482) or C4 in the following sequence CTC₂AC₄AATTAC₁₀AAC₁₃TGGGGCC (Site 2; SEQ ID NO: 483), respectively, in IVS26 lenti-integrated HEK293T cells. Guide RNAs were prepared with spacer lengths of 18 nt, 19 nt, 20 nt, 21 nt, 22 nt, or 23 nt to target the BE4 cytidine deaminase base editor to deaminase C9 at site 1 or C4 at site 2 (see Table 1 and FIGS. 7 and 8). The IVS26 lenti-integrated HEK293T cells were contacted with a plasmid encoding a guide RNA expressed under the control of a U6 promoter and a plasmid encoding the rAPOBEC BE4 cytidine deaminase base editor expressed under the control of a CMV promoter.

At Site 1 (FIG. 7), editing occurred at the disease allele (C5) and a bystander edit was observed at C7 (i.e., C5T). At Site 2 (FIG. 8), a favorable base editing profile was observed with editing taking place primarily at the target C4 site and at the C2 site. At Site 2 (FIG. 8), the 19 nucleotide guide had editing efficiency of ˜35% at the desired cryptic splice donor site with minimal bystander editing.

The above-described Examples demonstrate the feasibility of using cytidine base editors to treat Leber's Congenital Amaurosis-10 through correcting the pathogenic IVS26 mutation back to the wild type form, and/or through disruption of a splice donor site near the IVS26 mutation. Both of these alterations can prevent missplicing of mRNA transcribed from the CEP290 gene. In embodiments, an adenosine deaminase base editor is used to disrupt the splice donor site. Cytidine deaminase base editors (e.g., ppAPOBEC) were used in combination with length-optimized guide RNAs to reach editing efficiencies as high as 75% for correcting the IVS26 mutation. Cytidine deaminase base editors were used in combination with length-optimized guide RNAs to disrupt the splice site with editing efficiencies of up to 35% with minimal or no detectable bystander edits.

The following materials and methods were employed in the above examples.

### Lenti-Integrated HEK293T Cells

Leber's Congenital Amaurosis-10 (LCA10) is associated with a CEP290 c.2991+1655A>G mutation (the IVS26 mutation), which results in the introduction of a cryptic splice donor site within an intron of CEP290. The cryptic splice donor site results in the retention of a ˜128 base pair sequence from an intron of the CEP290 following splicing of an mRNA transcript transcribed from the mutated CEP290 gene (FIGS. 1A and 1B). A ˜300 bp region of interest (ROI) containing the following nucleotide sequence gggtttcaccttgttagccaggatggtgtcgatctcctgaactcgtgatccacccgcctcgg cctcctaaagtgctgggattaCAGATGTGAGccaccGCACCTGGCCCCAGTTGTAATTGTGA gTATCTCATACCTATCCCTATTGGCAGTGTCTTAGTTTTATTTTTTATTATCTTTATTGTGG CAGCCATTATTCCTGTCTCTATCTCCAGTCTTACATCCTCCTTACTGCCACAAGAATGATCA TT, where the pathogenic IVS26 mutation is in boldface and underlined; SEQ ID NO: 488, was randomly integrated into the genomes of HEK293T cells (FIGS. 3A and 3B) to prepare IVS26 lenti-integrated HEK293T cells suitable for use in evaluating base editing to treat LCA10. In the HEK293T cells, base editing was used to correct the IVS26 mutation using a cytidine base editor to target the C complementary to the pathogenic G base (FIGS. 2A and 3A), causing a reversion to the wild type A (FIGS. 2A and 3A). Additionally, in the IVS26 lenti-integrated HEK293T cells, 3 and 4 base pairs upstream of the IVS26 mutation, there was a “GT” splice donor site, that corresponds to a cryptic splice donor site that results in retention of the ˜128 base pair sequence following transcript splicing in subjects with LCA10 (FIGS. 2B, 3A and 3B). A and C bases complementary to this “GT” splice donor were targeted using cytidine deaminase base editors to disrupt the splice donor site. Cytidine deaminase base editors, adenosine deaminase base editors, and/or CABEs can be used to disrupt the splice donor site.

### Transfection and Base Editing

CEP290 c.2991+1655A>G (IVS26) lenti-integrated HEK293T cells were seeded at a density of 40,000 cells per well in a 48 well plate. All transfections were performed in triplicate or quadruplicate using Lipofectamine 2000 (1.5 μL of reagent per 1 μg of DNA transfected). For each transfection 750 ng of editor DNA and 250 ng of guide RNA plasmid. Reagent mixtures, which included plasmids encoding a base editor (BE4) and a guide polynucleotide, were added to the wells. Media was replaced every 48 hours over a 5-day (120 hour) period and cells were then lysed. Editing was measured 5 days post transfection. For lysing the cells, the media was removed and cell lysis buffer was added to the wells (10 mM Tris HCl (pH 8.0)+0.05% SDS+100 μg/mL Proteinase K) in an amount sufficient for gDNA extraction to allow for sequencing of a genomic site of interest. The plate was incubated at 55° C. for 1 hour and was then heat inactivated at 95° C. for 20 minutes. Samples were then stored in the −20° C. freezer.

### Sequencing

Cell lysate (2 μL) or cDNA was added to a 25 μL PCR reaction containing Q5 Hot Start HiFi 2× Master Mix and 0.5 μM of each primer containing 5′ Illumina adapter overhangs. PCR reactions were carried out as follows: 95° C. for 2 min, 30 cycles of (95° C. for 15 s, 65° C. for 20 s, and 72° C. for 20 s), and a final 72° C. extension for 2 min. Following amplification, 2 μL of the crude PCR products containing an amplified genomic site of interest were barcoded using 0.5 μM of each unique Illumina barcoding primer pairs and Q5 Hot Start High-Fidelity 2× Master Mix in a total volume of 25 μL. The reactions were carried out as follows: 98° C. for 2 min, 10 cycles of (98° C. for 20 s, 60° C. for 30 s, and 72° C. for 30 s), and a final 72° C. extension for 2 min. Equal volumes of barcoded PCR products were then pooled and cleaned up using SPRISelect paramagnetic beads (Beckman Coulter) using a 0.6× bead/sample ratio. Eluted DNA concentration was quantified with a Qubit 4 (Thermo Fisher Scientific) and was sequenced with an Illumina MiSeq instrument according to the manufacturer's protocol.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed:

1. A method of editing a nucleobase of a 290-KD centrosomal protein (CEP290) polynucleotide in a cell, the method comprising contacting the cell with:
(a) a base editor polypeptide comprising a nucleic acid programmable DNA binding protein (napDNAbp) domain and a deaminase domain, or
(b) one or more polynucleotides encoding the base editor, and one or more guide polynucleotides, or one or more polynucleotides encoding the one or more guide polynucleotides, wherein the one or more guide polynucleotides target said base editor to effect an alteration of the nucleobase of the CEP290 polynucleotide in the cell.

2. The method of claim 1, wherein the cell is in a subject having Leber's Congenital Amaurosis-10 (LCA10), and the alteration of the nucleobase of the 290-KD centrosomal protein (CEP290) polynucleotide in the cell treats LCA10 in the subject.

3. The method of claim 1, wherein alteration of the nucleobase is associated with any one or more of the following:
an alteration in splicing
an increase in proper splicing of mRNA transcripts transcribed from the CEP290 polynucleotide;
an increase in levels of functional CEP290 polypeptides in the cell;
alleviating one or more symptoms of LCA10 in the subject;
slowing or halting progression of vision loss associated with LCA10 in the subject;
reducing loss of functional rod and/or cone cells associated with LCA10 in the subject.

4. The method of claim 1, wherein the base editor effects an alteration of a nucleobase selected from the group consisting of CEP290 c.2991+1651, CEP290 c.2991+1652, and CEP290 c.2991+1655.

5. The method of claim 4, wherein the base editor effects an alteration of a nucleobase selected from the group consisting of
CEP290 c.2991+1655G>A;
CEP290 c.2991+1652T>C; and
CEP290 c.2991+1652G>A;

6. The method of claim 1, wherein the one or more guide polynucleotides comprise a nucleic acid sequence comprising at least 10 contiguous nucleotides of a spacer corresponding to a nucleic acid sequence selected from the group consisting of:
(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGAUACU
CACAAUUACAAC;
(SEQ ID NO: 466)
AUCUCACUGAUGAGUCCGUGAGGACGAAACGAGUAAGCUCGUCUGAGAU
ACUCACAAUUACAAC;
(SEQ ID NO: 467)
UAUCUCAUCUGAUGAGUCCGUGAGGACGAAACGAGUAAGCUCGUCAUGA
GAUACUCACAAUUACAAC;
(SEQ ID NO: 468)
CUCAUACUGAUGAGUCCGUGAGGACGAAACGAGUAAGCUCGUCUAUGAG
AUACUCACAAUUACAAC;
(SEQ ID NO: 469)
ACUCACAAUUACAACUG;
(SEQ ID NO: 470)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC;
wherein the one or more guide polynucleotides comprise a scaffold comprising a nucleotide sequence with at least about 85% sequence identity to the following sequence:
(SEQ ID NO: 484)
GUUUUAGAGCUAGAAAUAGCAAGUUAAAAUAAGGCUAGUCCGUUAUCAA
CUUGAAAAAGUGGCACCGAGUCGGUGCUUUUUU;
wherein the one or more guide polynucleotides comprise one or more of a 2′-OMe and a phosphorothioate.

7. The method of claim 1, further comprising contacting the cell with a vector comprising polynucleotide(s) encoding the base editor and/or the one or more guide polynucleotides.

8. The method of claim 1, wherein the vector is a lipid nanoparticle or an adeno-associated virus (AAV) vector.

9. The method of claim 1, wherein the polynucleotide(s) encoding the base editor and/or one or more guide polynucleotides comprises a promoter controlling expression of the base editor and/or one or more guide polynucleotides, wherein the promoter is selected from the group consisting of CMV, PR1.7, hG1.7, hGRK, and U6.

10. The method of claim 1, wherein the promoter is selected from the group consisting of PR1.7, hG1.7, and hGRK.

11. The method of claim 1, wherein the deaminase domain is an adenosine deaminase, a cytidine deaminase domain, or a cytidine adenosine deaminase domain.

12. A modified cell comprising an alteration in a nucleobase of a CEP290 polynucleotide, wherein the alteration increases expression and/or activity of the encoded CEP290 polypeptide as compared to a control cell without the alteration, and wherein the cell is prepared according to the method of claim 1.

13. A base editor system comprising one or more polynucleotides encoding a base editor polypeptide, wherein the base editor polypeptide comprises a nucleic acid programmable DNA binding protein (napDNAbp) domain and a deaminase domain, and one or more guide polynucleotides, or one or more polynucleotides encoding the one or more guide polynucleotides, wherein the one or more guide polynucleotides comprise a spacer sequence comprising at least 10 contiguous nucleotides of a spacer corresponding to a nucleotide sequence selected from the group consisting of:
(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGAUACU
CACAAUUACAAC;
(SEQ ID NO: 466)
AUCUCACUGAUGAGUCCGUGAGGACGAAACGAGUAAGCUCGUCUGAGAU
ACUCACAAUUACAAC;
(SEQ ID NO: 467)
UAUCUCAUCUGAUGAGUCCGUGAGGACGAAACGAGUAAGCUCGUCAUGA
GAUACUCACAAUUACAAC;
(SEQ ID NO: 468)
CUCAUACUGAUGAGUCCGUGAGGACGAAACGAGUAAGCUCGUCUAUGAG
AUACUCACAAUUACAAC;
(SEQ ID NO: 469)
ACUCACAAUUACAACUG;
(SEQ ID NO: 470)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

14. A set of one or more polynucleotides encoding the base editor system of claim 13, or a component thereof.

15. A vector comprising the set of one or more polynucleotides of claim 14.

16. The vector of claim 15, wherein the vector comprises a lipid nanoparticle or a viral vector.

17. A kit comprising a base editor system comprising a base editor polypeptide, or one or more polynucleotides encoding the same, wherein the base editor polypeptide comprises a nucleic acid programmable DNA binding protein (napDNAbp) domain and a deaminase domain, and one or more guide polynucleotides, or one or more polynucleotides encoding the one or more guide polynucleotides, wherein the one or more guide polynucleotides comprise a nucleotide sequence selected from the group consisting of:
(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGAUACU
CACAAUUACAAC;
(SEQ ID NO: 466)
AUCUCACUGAUGAGUCCGUGAGGACGAAACGAGUAAGCUCGUCUGAGAU
ACUCACAAUUACAAC;
(SEQ ID NO: 467)
UAUCUCAUCUGAUGAGUCCGUGAGGACGAAACGAGUAAGCUCGUCAUGA
GAUACUCACAAUUACAAC;
(SEQ ID NO: 468)
CUCAUACUGAUGAGUCCGUGAGGACGAAACGAGUAAGCUCGUCUAUGAG
AUACUCACAAUUACAAC;
(SEQ ID NO: 469)
ACUCACAAUUACAACUG;
(SEQ ID NO: 470)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

18. The kit of claim 17, further comprising written instructions for the use of the kit in the treatment of Leber congenital amaurosis 10 (LCA10).

19. A pharmaceutical composition comprising an effective amount of a base editor system comprising
(a) a base editor polypeptide, wherein the base editor polypeptide comprises a nucleic acid programmable DNA binding protein (napDNAbp) domain and a deaminase domain, or
(b) one or more polynucleotides encoding the base editor polypeptide, and one or more guide polynucleotides, or one or more polynucleotides encoding the one or more guide polynucleotides, wherein the one or more guide polynucleotides comprise a nucleotide sequence selected from the group consisting of:
(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGAUACU
CACAAUUACAAC;
(SEQ ID NO: 466)
AUCUCACUGAUGAGUCCGUGAGGACGAAACGAGUAAGCUCGUCUGAGAU
ACUCACAAUUACAAC;
(SEQ ID NO: 467)
UAUCUCAUCUGAUGAGUCCGUGAGGACGAAACGAGUAAGCUCGUCAUGA
GAUACUCACAAUUACAAC;
(SEQ ID NO: 468)
CUCAUACUGAUGAGUCCGUGAGGACGAAACGAGUAAGCUCGUCUAUGAG
AUACUCACAAUUACAAC;
(SEQ ID NO: 469)
ACUCACAAUUACAACUG;
(SEQ ID NO: 470)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.

20. A guide polynucleotide, or a polynucleotide encoding the guide polynucleotide, wherein the guide polynucleotide comprises a nucleotide sequence selected from the group consisting of:
(SEQ ID NO: 438)
GAUACUCACAAUUACAACGUUUUAGAGCUAGAAAUAGCAAGUUAAAAUAAGGCUAGUCCGUU
AUCAACUUGAAAAAGUGGCACCGAGUCGGUGCUUUUUU;
(SEQ ID NO: 439)
gGAUACUCACAAUUACAACGUUUUAGAGCUAGAAAUAGCAAGUUAAAAUAAGGCUAGUCCGU
UAUCAACUUGAAAAAGUGGCACCGAGUCGGUGCUUUUUU;
(SEQ ID NO: 440)
GAGAUACUCACAAUUACAACGUUUUAGAGCUAGAAAUAGCAAGUUAAAAUAAGGCUAGUCCG
UUAUCAACUUGAAAAAGUGGCACCGAGUCGGUGCUUUUUU;
(SEQ ID NO: 441)
gGAGAUACUCACAAUUACAACGUUUUAGAGCUAGAAAUAGCAAGUUAAAAUAAGGCUAGUCC
GUUAUCAACUUGAAAAAGUGGCACCGAGUCGGUGCUUUUUU;
(SEQ ID NO: 442)
gUGAGAUACUCACAAUUACAACGUUUUAGAGCUAGAAAUAGCAAGUUAAAAUAAGGCUAGUC
CGUUAUCAACUUGAAAAAGUGGCACCGAGUCGGUGCUUUUUU;
(SEQ ID NO: 443)
gAUGAGAUACUCACAAUUACAACGUUUUAGAGCUAGAAAUAGCAAGUUAAAAUAAGGCUAGU
CCGUUAUCAACUUGAAAAAGUGGCACCGAGUCGGUGCUUUUUU;
(SEQ ID NO: 444)
GUAUCUCUGAUGAGUCCGUGAGGACGAAACGAGUAAGCUCGUCAGAUACUCACAAUUACAAC
GUUUUAGAGCUAGAAAUAGCAAGUUAAAAUAAGGCUAGUCCGUUAUCAACUUGAAAAAGUGG
CACCGAGUCGGUGCUUUUUU;
(SEQ ID NO: 445)
gAUCUCACUGAUGAGUCCGUGAGGACGAAACGAGUAAGCUCGUCUGAGAUACUCACAAUUAC
AACGUUUUAGAGCUAGAAAUAGCAAGUUAAAAUAAGGCUAGUCCGUUAUCAACUUGAAAAAG
UGGCACCGAGUCGGUGCUUUUUU;
(SEQ ID NO: 446)
gUAUCUCAUCUGAUGAGUCCGUGAGGACGAAACGAGUAAGCUCGUCAUGAGAUACUCACAAU
UACAACGUUUUAGAGCUAGAAAUAGCAAGUUAAAAUAAGGCUAGUCCGUUAUCAACUUGAAA
AAGUGGCACCGAGUCGGUGCUUUUUU;
(SEQ ID NO: 447)
gCUCAUACUGAUGAGUCCGUGAGGACGAAACGAGUAAGCUCGUCUAUGAGAUACUCACAAUU
ACAACGUUUUAGAGCUAGAAAUAGCAAGUUAAAAUAAGGCUAGUCCGUUAUCAACUUGAAAA
AGUGGCACCGAGUCGGUGCUUUUUU;
(SEQ ID NO: 448)
gACUCACAAUUACAACUGGUUUUAGAGCUAGAAAUAGCAAGUUAAAAUAAGGCUAGUCCGUU
AUCAACUUGAAAAAGUGGCACCGAGUCGGUGCUUUUUU;
(SEQ ID NO: 449)
gUACUCACAAUUACAACUGGUUUUAGAGCUAGAAAUAGCAAGUUAAAAUAAGGCUAGUCCGU
UAUCAACUUGAAAAAGUGGCACCGAGUCGGUGCUUUUUU;
(SEQ ID NO: 450)
GAUACUCACAAUUACAACUGGUUUUAGAGCUAGAAAUAGCAAGUUAAAAUAAGGCUAGUCCG
UUAUCAACUUGAAAAAGUGGCACCGAGUCGGUGCUUUUUU;
(SEQ ID NO: 451)
gGAUACUCACAAUUACAACUGGUUUUAGAGCUAGAAAUAGCAAGUUAAAAUAAGGCUAGUCC
GUUAUCAACUUGAAAAAGUGGCACCGAGUCGGUGCUUUUUU;
(SEQ ID NO: 452)
GAGAUACUCACAAUUACAACUGGUUUUAGAGCUAGAAAUAGCAAGUUAAAAUAAGGCUAGUC
CGUUAUCAACUUGAAAAAGUGGCACCGAGUCGGUGCUUUUUU;
(SEQ ID NO: 453)
gCAAUUACAACUGGGGCCGUUUUAGAGCUAGAAAUAGCAAGUUAAAAUAAGGCUAGUCCGUU
AUCAACUUGAAAAAGUGGCACCGAGUCGGUGCUUUUUU;
(SEQ ID NO: 454)
gACAAUUACAACUGGGGCCGUUUUAGAGCUAGAAAUAGCAAGUUAAAAUAAGGCUAGUCCGU
UAUCAACUUGAAAAAGUGGCACCGAGUCGGUGCUUUUUU;
(SEQ ID NO: 455)
gCACAAUUACAACUGGGGCCGUUUUAGAGCUAGAAAUAGCAAGUUAAAAUAAGGCUAGUCCG
UUAUCAACUUGAAAAAGUGGCACCGAGUCGGUGCUUUUUU;
(SEQ ID NO: 456)
gUCACAAUUACAACUGGGGCCGUUUUAGAGCUAGAAAUAGCAAGUUAAAAUAAGGCUAGUCC
GUUAUCAACUUGAAAAAGUGGCACCGAGUCGGUGCUUUUUU;
(SEQ ID NO: 457)
gCUCACAAUUACAACUGGGGCCGUUUUAGAGCUAGAAAUAGCAAGUUAAAAUAAGGCUAGUC
CGUUAUCAACUUGAAAAAGUGGCACCGAGUCGGUGCUUUUUU;
(SEQ ID NO: 458)
gACUCACAAUUACAACUGGGGCCGUUUUAGAGCUAGAAAUAGCAAGUUAAAAUAAGGCUAGU
CCGUUAUCAACUUGAAAAAGUGGCACCGAGUCGGUGCUUUUUU;
(SEQ ID NO: 459)
AUACUCACAAUUACAAC;
(SEQ ID NO: 460)
GAUACUCACAAUUACAAC;
(SEQ ID NO: 461)
AGAUACUCACAAUUACAAC;
(SEQ ID NO: 462)
GAGAUACUCACAAUUACAAC;
(SEQ ID NO: 463)
UGAGAUACUCACAAUUACAAC;
(SEQ ID NO: 464)
AUGAGAUACUCACAAUUACAAC;
(SEQ ID NO: 465)
UAUCUCUGAUGAGUCCGUGAGGACGAAACGAGUAAGCUCGUCAGAUACU
CACAAUUACAAC;
(SEQ ID NO: 466)
AUCUCACUGAUGAGUCCGUGAGGACGAAACGAGUAAGCUCGUCUGAGAU
ACUCACAAUUACAAC;
(SEQ ID NO: 467)
UAUCUCAUCUGAUGAGUCCGUGAGGACGAAACGAGUAAGCUCGUCAUGA
GAUACUCACAAUUACAAC;
(SEQ ID NO: 468)
CUCAUACUGAUGAGUCCGUGAGGACGAAACGAGUAAGCUCGUCUAUGAG
AUACUCACAAUUACAAC;
(SEQ ID NO: 469)
ACUCACAAUUACAACUG;
(SEQ ID NO: 470)
UACUCACAAUUACAACUG;
(SEQ ID NO: 471)
AUACUCACAAUUACAACUG;
(SEQ ID NO: 472)
GAUACUCACAAUUACAACUG;
(SEQ ID NO: 473)
AGAUACUCACAAUUACAACUG;
(SEQ ID NO: 474)
CAAUUACAACUGGGGCC;
(SEQ ID NO: 475)
ACAAUUACAACUGGGGCC;
(SEQ ID NO: 476)
CACAAUUACAACUGGGGCC;
(SEQ ID NO: 477)
UCACAAUUACAACUGGGGCC;
(SEQ ID NO: 478)
CUCACAAUUACAACUGGGGCC;
and
(SEQ ID NO: 479)
ACUCACAAUUACAACUGGGGCC.
